(19) United States
(12) Patent Application Publication
(10) Pub. No.: US 2026/0081023 A1
(43) Pub. Date: Mar. 19, 2026

(54) METHOD AND SYSTEM FOR RISK STRATIFICATION AND CHEMOTHERAPY RESISTANCE PREDICTION IN PANCREATIC DUCTAL ADENOCARCINOMA

(71) Applicant: OHIO STATE INNOVATION FOUNDATION, Columbus, OH (US)

(72) Inventor: Ashish MANNE, Columbus, OH (US)

(21) Appl. No.: 19/328,400

(22) Filed: Sep. 15, 2025

Related U.S. Application Data
(60) Provisional application No. 63/694,456, filed on Sep. 13, 2024.

(51) Int. Cl. G16H 50/20; G16B 30/00; G16B 40/20; G16H 50/70

(52) U.S. Cl. CPC G16H 50/20; G16B 30/00; G16B 40/20; G16H 50/70

(57) ABSTRACT

An exemplary system and method for predicting treatment-related outcomes of patients after a cancer therapy and/or treatment (e.g., PDA treatment) using DNA (e.g., cell-free DNA (cfDNA)) or RNA methylation signatures and/or an RNA sequencing signature as predictive biomarkers for treatment response and overall survival in the patients.

### RELATED APPLICATION

The U.S. patent application claims priority to, and the benefit of, U.S. Provisional Patent Application No. 63/694,456, filed Sep. 13, 2024, entitled “Machine Learning-based Analysis and Personalized Models to Diagnose and Manage Disease with Cell-free DNA and Serum Proteins with Imaging,” which is incorporated by reference herein in its entirety.

### BACKGROUND

Pancreatic ductal adenocarcinoma (PDA) is an aggressive form of pancreatic cancer, with high mortality and limited early symptoms. PDA arises from the epithelial cells of the pancreatic ducts and is often diagnosed at an advanced stage. Its lethality comes from a high frequency of driver mutations, a dense and fibrotic tumor microenvironment that impairs drug delivery and immune cell infiltration, and the presence of cancer-associated fibroblasts (CAFs) that promote tumor growth and resistance to prediction and therapy. These biological complexities contribute to poor prognosis and limited therapeutic success, making PDA a focus of cancer research and therapy development.

There is a benefit to improving the system and method for predicting treatment-related outcomes of patients after cancer therapy, including PDA therapy.

### SUMMARY

An exemplary system and method are disclosed for predicting treatment-related outcomes of patients after a cancer therapy and/or treatment (e.g., PDA treatment) using DNA (e.g., cell-free DNA (cfDNA)) or RNA methylation signatures and/or RNA sequencing signatures as predictive biomarkers for treatment response and overall survival in the patients.

Different from current predictive systems that rely on mutational or transcriptomic data and often lack actionable targets, the exemplary system and method utilize DNA-based (e.g., cfDNA-based) or RNA-based epigenetic signatures to infer protein activity through methylation levels. The approach can provide the identification of gene signatures that not only stratify patients into high- and low-risk groups but also provide actionable insights into treatment response and overall survival (OS). Furthermore, the exemplary system and method employ trained artificial intelligence (AI) models that integrate clinical variables with methylation data to predict additional outcomes such as duration of response (DoR), progression-free survival (PFS), and time-to-progression (TTP).

By providing a non-invasive, DNA-based (e.g., cfDNA-based) or RNA-based predictive method with actionable gene targets, the exemplary system and method represent an advancement over current predictive systems, facilitating more personalized and effective treatment strategies for PDA patients.

In an aspect, a system for predicting a treatment-related outcome for a patient after a cancer therapy (e.g., chemotherapy), including an overall survival outcome, is disclosed comprising: a processor; and a memory having instructions stored thereon, wherein execution of the instructions causes the processor to: receive, via the processor, a methylation signature including methylated nucleic acid sequences (e.g., DNA, cell-free DNA (cfDNA), or RNA) or Ribonucleic acid (RNA) sequencing signature acquired from a sample (e.g., blood or tissue) of a patient for at least one gene selected from the group consisting of BNIP3, CES2, CHFR, CXCL5, GSTM2, ITGB4, MUC4, MUC5AC, ONECUT2, PRMT1, RUNX1, SFN, SLC22A3, SOX8, TACC3, KCNH2, PROKR2, IGF1R, and TMEM139; determine, via a trained AI model, using the received methylated sequences or RNA sequences, an indicator corresponding to an overall survival outcome of the patient from pancreatic cancer and/or associated treatments; and output the determined indicator via a report or graphical user interface, wherein the output is subsequently employed to direct or adjust treatment of the pancreatic cancer for the patient.

In some embodiments, the trained AI model was trained using methylated sequences or RNA sequences for a plurality of genes, including at least 5 of BNIP3, CES2, CHFR, CXCL5, GSTM2, ITGB4, MUC4, MUC5AC, ONECUT2, PRMT1, RUNX1, SFN, SLC22A3, SOX8, TACC3, KCNH2, PROKR2, IGF1R, TMEM139, wherein the methylation signature or RNA sequencing signature is stratified for a patient population having a high risk group label and a lower risk group label for overall survival.

In some embodiments, the overall survival is determined at 6 months, 1 year, or 2 years from the date of diagnosis of the pancreatic cancer.

In some embodiments, execution of the instructions further causes the processor to additionally predict at least one of a predicted duration of response, a predicted progression-free survival time, and a predicted time to progression.

In some embodiments, the instructions to determine the additional prediction for the at least one of the predicted duration of response, the predicted progression-free survival time, and the predicted time to progression includes: instructions to determine, via a second trained AI model, the received methylated sequences or RNA sequences for at least one gene selected from the group consisting of BNIP3, CES2, CHFR, CXCL5, GSTM2, ITGB4, MUC4, ONECUT2, PRMT1, RUNX1, SFN, SLC22A3, SOX8, TACC3, IGF1R, KCNH2, MUC5AC, SLC22A2, SST, TMEM139, ISG15, PROKR2, SLC38A5, and SMARCA2.

In some embodiments, the instructions to determine the additional prediction for the predicted duration of response, includes: instructions to determine, via a second trained AI model, the received methylated sequences or RNA sequences for at least one gene (e.g., 50-75% of the genes in the list) in a gene selected from the group consisting of BNIP3, CES2, CHFR, CXCL5, GSTM2, ITGB4, MUC4, ONECUT2, PRMT1, RUNX1, SFN, SLC22A3, SOX8, TACC3, IGF1R, KCNH2, MUC5AC, SST, and TMEM139.

In some embodiments, the instructions to determine the additional prediction for the predicted progression-free survival time includes: instructions to determine, via a second trained AI model, the received methylated sequences or RNA sequences for at least one gene (e.g., 50-75% of the genes in the list) in a gene selected from the group consisting of BNIP3, CES2, IGF1R, ISG15, ITGB4, KCNH2, ONECUT2, PROKR2, RUNX1, SFN, SLC22A3, SLC38A5, SMARCA2, SOX8, SST, and TACC3.

In some embodiments, the instructions to determine the additional prediction for the predicted time to progression includes: instructions to determine, via a second trained AI model, the received methylated sequences or RNA sequences for at least one gene (e.g., 50-75% of the genes in the list) in a gene selected from the group consisting of BNIP3, CES2, CHFR, CXCL5, GSTM2, IGF1R, ISG15, ITGB4, KCNH2, MUC4, ONECUT2, PROKR2, RUNX1, SFN, SLC22A3, SLC38A5, SMARCA2, SOX8, SST, and TACC3.

In some embodiments, the predicted duration of response, the predicted progression-free survival time, and the predicted time to progression is determined at 6 months, 1 year, or 2 years from a date of diagnosis or a date of initial treatment.

In some embodiments, the trained AI model is a convolutional neural network.

In some embodiments, the methylated sequences or RNA sequences were acquired via a sequencing operation.

In some embodiments, the sequencing operation includes an Enzymatic Methylation Sequencing operation.

In some embodiments, the sample includes blood plasma and/or tissues.

In some embodiments, pancreatic cancer includes pancreatic ductal adenocarcinoma (PDA).

In some embodiments, the instructions to determine the additional prediction for the at least one of the predicted duration of response, the predicted progression-free survival time, and the predicted time to progression includes: instructions to determine, via a second trained AI model, the received methylated sequences or RNA sequences for at least one gene selected from the group consisting of ABCB1, ABCB4, ABCC1, ABCC10, ABCC3, ABCC5, ABCC6, ABCC8, ABCC9, ABCG2, ANGPTL4, ARID1A, ASXL2, ATM, BCL2L1, BICC1, BNIP3, BRCA1, CADM1, CD44, CES2, CHFR, CTNNB1, CTPS2, CXCL5, DCK, DKK3, DPYD, EGFR, EIF5A, ENO1, GLO1, GSDME, GSTM1, GSTM2, HMGA1, HNF1A, HSPA5, HSPB1, IGF1R, IGFBP3, ISG15, ITGA3, ITGB4, JAG1, KCNH2, LDHA, MAP2, MAP3K7, MCL1, METTL3, MLH1, MUC4, MUC5AC, NOTCH2, NRP1, NT5C1A, ONECUT2, PRMT1, PROKR2, PTGES2, PYCARD, RELL2, RRM1, RRM2, RRP9, RUNX1, SFN, SLC22A2, SLC22A3, SLC29A1, SLC2A1, SLC38A5, SMARCA2, SNRPF, SOX8, SST, TACC3, TET1, TFAM, TGM2, TMEM139, TPX2, TRIM31, TYMS, UBE2T, USP8, VASH2, YEATS4, and ZEB1.

In some embodiments, the trained AI model was trained using a methylation signature including the methylated sequences from an isolated nucleic acid (e.g., DNA, cfDNA, RNA) from the sample (e.g., plasma of a patient).

In another aspect, a method for predicting a treatment-related outcome for a patient after a cancer therapy (e.g., chemotherapy), including an overall survival outcome, is disclosed comprising: receiving, via a processor, a methylation signature including methylated nucleic acid sequences (e.g., DNA, cfDNA, RNA) or RNA sequencing signature acquired from a sample (e.g., blood or tissue) of a patient for at least one gene selected from the group consisting of BNIP3, CES2, CHFR, CXCL5, GSTM2, ITGB4, MUC4, MUC5AC, ONECUT2, PRMT1, RUNX1, SFN, SLC22A3, SOX8, TACC3, KCNH2, PROKR2, IGF1R, and TMEM139; determining, via a trained AI model, using the received methylated sequences or RNA sequences, an indicator corresponding to an overall survival outcome of the patient from pancreatic cancer and/or associated treatments; and outputting the determined indicator via a report or graphical user interface, wherein the output is subsequently employed to direct or adjust treatment of the pancreatic cancer for the patient.

In some embodiments, the trained AI model was trained using methylated sequences or RNA sequences for a plurality of genes, including at least 5 of BNIP3, CES2, CHFR, CXCL5, GSTM2, ITGB4, MUC4, MUC5AC, ONECUT2, PRMT1, RUNX1, SFN, SLC22A3, SOX8, TACC3, KCNH2, PROKR2, IGF1R, TMEM139, wherein the methylation signature or RNA sequencing signature is stratified for a patient population having a high risk group label and a lower risk group label for overall survival.

In some embodiments, the overall survival is determined at 6 months, 1 year, or 2 years from date of diagnosis of the pancreatic cancer.

In some embodiments, a non-transitory computer-readable medium having instructions stored thereon is disclosed, wherein execution of the instructions causes a processor to: receive, via the processor, a methylation signature including methylated nucleic acid sequences (e.g., DNA, cfDNA, RNA) or RNA sequencing signature acquired from a sample (e.g., blood or tissue) of a patient for at least one gene selected from the group consisting of BNIP3, CES2, CHFR, CXCL5, GSTM2, ITGB4, MUC4, MUC5AC, ONECUT2, PRMT1, RUNX1, SFN, SLC22A3, SOX8, TACC3, KCNH2, PROKR2, IGF1R, and TMEM139; determine, via a trained AI model, using the received methylated sequences, an indicator corresponding to an overall survival outcome of the patient from pancreatic cancer and/or associated treatments; and output the determined indicator via a report or graphical user interface, wherein the output is subsequently employed to direct or adjust treatment of the pancreatic cancer for the patient.

### BRIEF DESCRIPTION OF DRAWINGS

FIGS. 1A-1B each shows an example system for predicting a treatment-related outcome for a patient after a cancer therapy (e.g., chemotherapy), in accordance with an illustrative embodiment.

FIGS. 2A-2D each shows an example training process for training the artificial intelligence (AI) model of the exemplary system.

FIG. 3 shows an example sequence extraction operation configured to extract/synthesize a methylated sequence described in relation to FIGS. 1-2.

FIG. 4 shows an example operation flow for the exemplary system, in accordance with an illustrative embodiment.

FIG. 5A shows an example methodology for creating a cfDNA epigenetic panel of genes. FIG. 5B shows an example relationship between methylation changes and protein expression.

FIGS. 6A-6H show experimental procedures and results for evaluating an experimental system and method described in relation to FIGS. 1-4.

FIGS. 7A-7D show experimental AI models developed for the experimental system and method.

### DETAILED DESCRIPTION

Some references, which may include various patents, patent applications, and publications, are cited in a reference list and discussed in the disclosure provided herein. The citation and/or discussion of such references is provided merely to clarify the description of the disclosed technology and is not an admission that any such reference is “prior art” to any aspects of the disclosed technology described herein. In terms of notation, “[n]” corresponds to the nth reference in the list. For example, [1] refers to the first reference in the list. All references cited and discussed in this specification are incorporated herein by reference in their entirety and to the same extent as if each reference were individually incorporated by reference.

### Example System

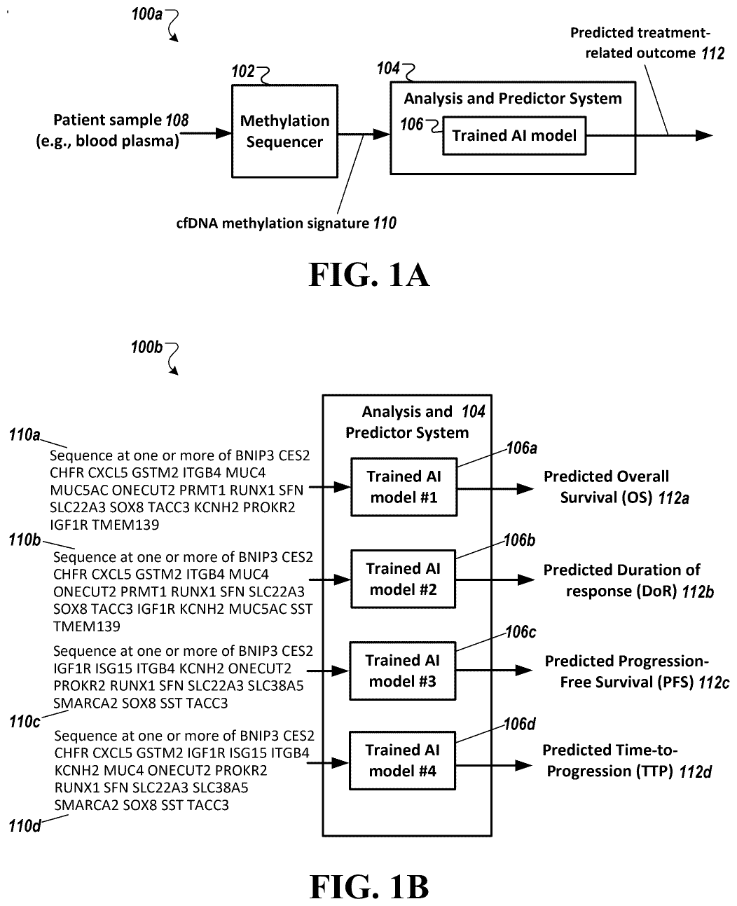

FIGS. 1A-1B each shows an example system 100 (shown as 100a, 100b) for predicting a treatment-related outcome for a patient after a cancer therapy (e.g., chemotherapy), in accordance with an illustrative embodiment. The exemplary system 100 can comprise (i) a methylation sequencer 102 configured to synthesize a cell-free DNA (cfDNA), DNA, or RNA methylation signature from a patient sample (e.g., blood plasma, tissue) and (ii) an analysis and predictor system 104 having a trained AI model configured to predict the treatment-related outcome, e.g., using indicator(s) (e.g., overall survival (OS), duration of response (DoR), progression-free survival (PFS), time-to-progression (TTP)), based on the cfDNA methylation signature. A “methylation signature” as defined herein describes the methylation state of one or more genomic sequences (e.g., genes), and in some embodiments refers to the characteristics of a nucleic acid segment at a particular genomic locus relevant to methylation. DNA methylation refers to the addition of a methyl group to the 5′ carbon of cytosine residues (i.e., 5-methylcytosines) among, e.g., CpG dinucleotides. DNA methylation may occur in cytosines in other contexts, for example CHG and CHH, where H is adenine, cytosine or thymine. Cytosine methylation may also be in the form of 5-hydroxymethylcytosine. Non-cytosine methylation, such as N6-methyladenine, has also been reported. In some embodiments, the term “methylation signature” refers to the relative or absolute concentration of methylated C or unmethylated C at any particular set or stretch of residues in a biological sample.

Methylation sequencing may be performed to evaluate or study DNA or RNA methylation patterns across the genome where a biological process adds methyl group (CH3) to the cytosine bases of the nucleic acid molecule. Methylation sequencing occurs at many sites for a gene and thus can be distinguished from mutations. Methylation levels may be averaged across locations within each gene. Genes may be filtered out if their average methylation level is less than 0.05. Other levels may be applied (e.g., 0.01, 0.02, 0.03, 0.04, 0.05, 0.06, 0.07, 0.08, 0.09, 0.10, etc.). For univariate analysis, methylation levels for all samples may be obtained to test differences between the groups for each gene. For multivariate analysis, predictive models may be employed, e.g., using Cox regression with a backward selection method. Important clinical variables are then adjusted. Risk scores for all samples may be calculated from the multivariate predictive models, and patients are then divided into high- and low-risk groups based on these scores. Kaplan-Meier curves may be plotted along with log-rank tests to compare differences between the two risk groups.

Analysis and Predictor System (104). In the example shown in FIG. 1A, the analysis and predictor system 104 is configured to receive, from the methylation sequencer 102, a DNA (e.g., cfDNA) or RNA methylation signature 110. The methylation signature 110 can comprise methylated nucleic acid sequences (e.g., DNA, cfDNA, RNA) or a Ribonucleic acid (RNA) sequencing signature acquired from a patient sample 108 (e.g., blood plasma, tissue) for at least one gene selected from the group consisting of ABCB1, ABCB4, ABCC1, ABCC10, ABCC3, ABCC5, ABCC6, ABCC8, ABCC9, ABCG2, ANGPTL4, ARID1A, ASXL2, ATM, BCL2L1, BICC1, BNIP3, BRCA1, CADM1, CD44, CES2, CHFR, CTNNB1, CTPS2, CXCL5, DCK, DKK3, DPYD, EGFR, EIF5A, ENO1, GLO1, GSDME, GSTM1, GSTM2, HMGA1, HNF1A, HSPA5, HSPB1, IGF1R, IGFBP3, ISG15, ITGA3, ITGB4, JAG1, KCNH2, LDHA, MAP2, MAP3K7, MCL1, METTL3, MLH1, MUC4, MUC5AC, NOTCH2, NRP1, NT5C1A, ONECUT2, PRMT1, PROKR2, PTGES2, PYCARD, RELL2, RRM1, RRM2, RRP9, RUNX1, SFN, SLC22A2, SLC22A3, SLC29A1, SLC2A1, SLC38A5, SMARCA2, SNRPF, SOX8, SST, TACC3, TET1, TFAM, TGM2, TMEM139, TPX2, TRIM31, TYMS, UBE2T, USP8, VASH2, YEATS4, and ZEB1. In some embodiments, the methylation signature 110 (and methylated and/or RNA sequences therein) is acquired via methods for detecting methylated nucleotide sequences, including but not limited to treating bisulfite methylation sequencing, methylation aware sequencing, or enzymatic methylation sequencing. For example, sodium bisulfite converts C, but not 5 mC, to U. Methods for bisulfite treatment of DNA are well-known in the art (Herman, et al., 1996, Proc Natl Acad Sci USA, 93:9821-6; Herman and Baylin, 1998, Current Protocols in Human Genetics, N. E. A. Dracopoli, ed., John Wiley & Sons, 2:10.6.1-10.6.10; U.S. Pat. No. 5,786,146). Methods of measuring a methylation signature, e.g., the level of methylation, may include, but are not limited to, massively parallel sequencing (e.g., next-generation sequencing) or sequencing real-time (e.g., single-molecule) sequencing, bead emulsion sequencing, nanopore sequencing, or other sequencing techniques known in the art. In some embodiments, assaying a methylation signature can include whole-genome sequencing, e.g., measuring whole genome methylation status from bisulfite or enzymatically treated material with base-pair resolution. Methylation-sensitive restriction enzymes that typically digest unmethylated DNA provide a low cost approach to study DNA methylation. Affinity capture or immunoprecipitation of DNA bound by anti-methylated cytosine antibodies can be used to survey large segments of the genome. In some embodiments, assaying a methylation signature in any aspect disclosed herein can include targeted sequencing, e.g., measuring methylation status of pre-selected gene from bisulfite or enzymatically treated material with base-pair resolution. When a nucleic acid molecule that contains unmethylated C nucleotides is treated with sodium bisulfite, the sequence of that DNA is changed (C→U). Detection of a U base in the converted nucleotide sequence is indicative of an unmethylated C, which can be detected by using, e.g., methylation sensitive PCR using methylation-specific primers. In some embodiments, the methylation signature 110 (and methylated and/or RNA sequences therein) is acquired via an Enzymatic Methylation Sequencing operation. Enzymatic Methylation Sequencing operations are known in the art (see, e.g., Vaisvila R et al. Enzymatic methyl sequencing detects DNA methylation at single-base resolution from picograms of DNA. Genome Res. 2021 July; 31(7):1280-1289, incorporated herein by reference for all purposes) involve detected 5mC and 5hmC using two sets of enzymatic reactions. In the first reaction, TET2 and T4-BGT convert 5mC and 5hmC into products that cannot be deaminated by APOBEC3A. In the second reaction, APOBEC3A deaminates unmodified C by conversion to U. Resulting product can then be analyzed by sequencing (e.g., next-generation Illumina sequencing). Therefore, these three enzymes enable the identification of 5mC and 5hmC. In some embodiments, the sample is selected from blood, plasma, serum, urine, sputum, spinal fluid, cerebrospinal fluid, pleural fluid, nipple aspirate, lymph fluid, respiratory tract fluid, intestinal tract fluid, genitourinary tract fluid, tear fluid, saliva, breast milk, lymphatic system fluid, semen, ascitic fluid, tumor cyst fluid, amniotic fluid, tissue, biopsy, or a combination thereof.

The analysis and predictor system 104 is then configured to determine, via the trained AI model 106, using the methylated sequences or the RNA sequencing signature in the nucleic acid methylation signature 110, an indicator 112 (shown as 112a-112d) corresponding to a predicted treatment-related outcome (e.g., OS, DoR, PFS, TTP) of the patient after the cancer therapy. The analysis and predictor system 104 is then configured to output the determined indicator 112 via a report or graphical user interface, where the output is subsequently employed to direct or adjust treatment of cancer (e.g., pancreatic cancer) for the patient.

In some embodiments, the predicted treatment-related outcome, expressed via an indicator 112, includes overall survival (OS), duration of response (DoR), progression-free survival (PFS) time, and time to progression (TTP), all of which are detailed in Table 2. In some embodiments, the predicted OS, the predicted DoR, the predicted PFS, and the predicted TTP are determined at 6 months, 1 year, or 2 years from a date of diagnosis or a date of initial treatment.

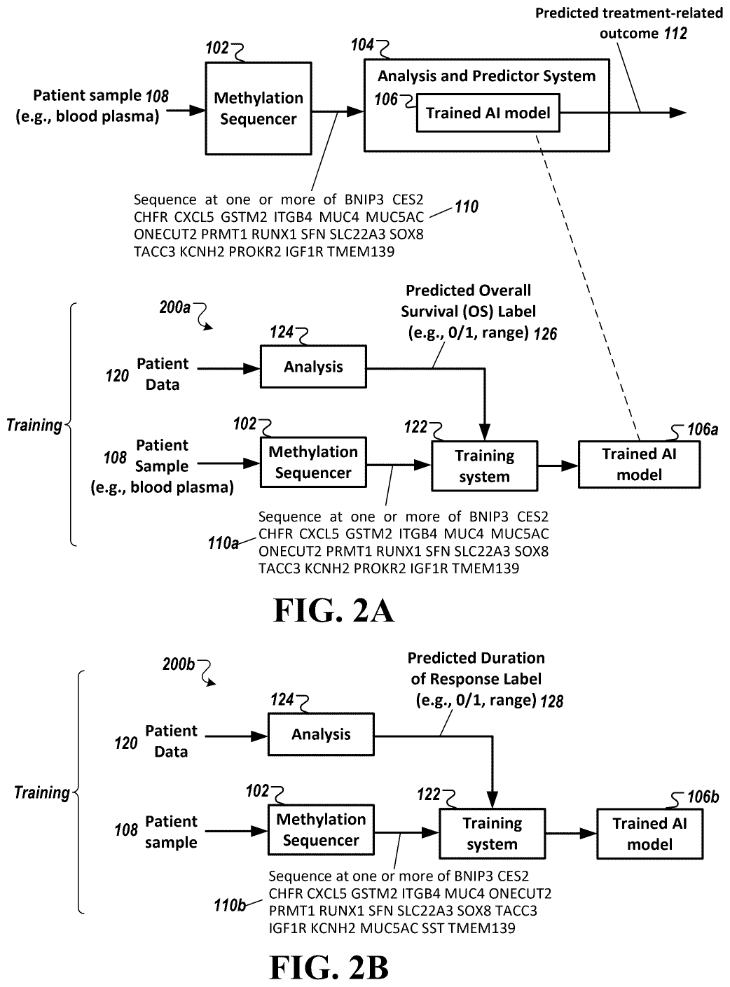

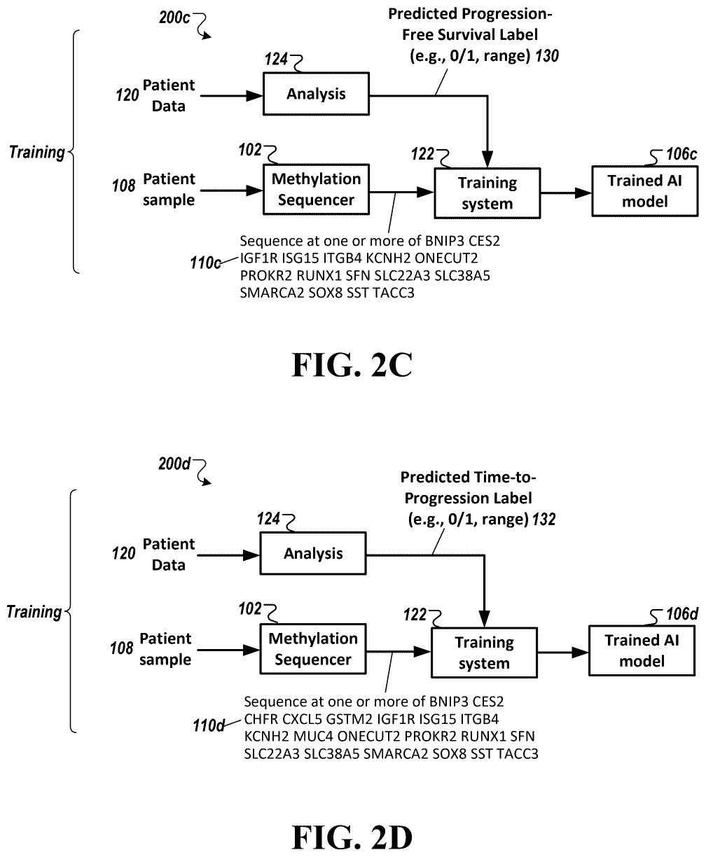

In the example shown in FIG. 1B, the system 100b can employ up to 4 trained AI models 106a-106d, each of which was trained, using different methylation sequences and labels (see FIGS. 2A-2D), to generate indicators corresponding to different predicted treatment-related outcomes (e.g., OS, DoR, PFS, TTP).

Specifically, the analysis and predictor system 104 is configured to determine, via the trained AI model 106a, using the received methylated sequence 110a for at least one gene (e.g., 50-75% of the genes in the group) selected from the group consisting of BNIP3, CES2, CHFR, CXCL5, GSTM2, ITGB4, MUC4, MUC5AC, ONECUT2, PRMT1, RUNX1, SFN, SLC22A3, SOX8, TACC3, KCNH2, PROKR2, IGF1R, and TMEM139, the indicator 112a corresponding to the predicted overall survival (OS) outcome of the patient from the cancer therapy (e.g., chemotherapy).

The analysis and predictor system 104 is also configured to determine, via the trained AI model 106b, using the received methylated sequence 110b for at least one gene (e.g., 50-75% of the genes in the group) selected from the group consisting of BNIP3, CES2, CHFR, CXCL5, GSTM2, ITGB4, MUC4, ONECUT2, PRMT1, RUNX1, SFN, SLC22A3, SOX8, TACC3, IGF1R, KCNH2, MUC5AC, SST, and TMEM139, the indicator 112b corresponding to the predicted Duration of Response (DoR) outcome of the patient from the cancer therapy.

The analysis and predictor system 104 is also configured to determine, via the trained AI model 106c, using the received methylated sequence 110c for at least one gene (e.g., 50-75% of the genes in the group) selected from the group consisting of BNIP3, CES2, IGF1R, ISG15, ITGB4, KCNH2, ONECUT2, PROKR2, RUNX1, SFN, SLC22A3, SLC38A5, SMARCA2, SOX8, SST, and TACC3, the indicator 112c corresponding to the predicted progression-free survival (PFS) outcome of the patient from the cancer therapy.

The analysis and predictor system 104 is also configured to determine, via the trained AI model 106d, using the received methylated sequence 110d for at least one gene (e.g., 50-75% of the genes in the group) selected from the group consisting of BNIP3, CES2, CHFR, CXCL5, GSTM2, IGF1R, ISG15, ITGB4, KCNH2, MUC4, ONECUT2, PROKR2, RUNX1, SFN, SLC22A3, SLC38A5, SMARCA2, SOX8, SST, and TACC3, the indicator 112d corresponding to the predicted time-to-progression (TTP) outcome of the patient from the cancer therapy.

### Example Artificial Intelligence (AI) and Machine Learning (ML) Model

FIGS. 2A-2D each shows an example training process 200 (shown as 200a-200d) for training the AI model 106 (e.g., 106a-106d) of the exemplary system 100. Each training process 200a-200d includes a training system 122 configured to (i) receive labels (referred to as ground truths) and methylated sequences and (ii) train the AI model 106 using the received labels and sequences. The trained AI model 106 (e.g., 106a-106d) is subsequently used by the analysis and predictor system 104 to predict the treatment-related outcome 112 of the patient from the cancer therapy (e.g., chemotherapy). As used herein, a methylated sequence refers to a cfDNA methylated sequence, a DNA methylated sequence, or a RNA methylated sequence. In some embodiments, the DNA methylated sequence is derived from a formalin-fixed paraffin-embedded (FFPE) DNA isolated from a biopsy or tissue sample.

In the example shown in FIG. 2A, the training system 122 is configured to receive (i) a methylated sequence 110a for a plurality of genes, including at least 5 of BNIP3, CES2, CHFR, CXCL5, GSTM2, ITGB4, MUC4, MUC5AC, ONECUT2, PRMT1, RUNX1, SFN, SLC22A3, SOX8, TACC3, KCNH2, PROKR2, IGF1R, and TMEM139 and (ii) a predicted overall survival (OS) label 126 (e.g., 0/1, range, high-risk, low-risk). The methylated sequence 110a is synthesized by the methylation sequencer 102 using the patient sample 108 (e.g., blood plasma, tissue). The predicted OS label 126 is generated by an analysis operation 124 using patient data 120 (e.g., medical/family history, demographics, etc).

The training system 122 is configured to train the AI model 106 using the received methylated sequence 110a and the predicted OS label 126. The resulting trained AI model 106a is subsequently used by the analysis and predictor system 104 to predict the overall survival outcome 112a.

In FIG. 2B, the training system 122 is configured to receive (i) a methylated sequence 110b for a plurality of genes, including at least 5 of BNIP3, CES2, CHFR, CXCL5, GSTM2, ITGB4, MUC4, ONECUT2, PRMT1, RUNX1, SFN, SLC22A3, SOX8, TACC3, IGF1R, KCNH2, MUC5AC, SST, and TMEM139, and (ii) a predicted Duration of Response (DoR) label 128 (e.g., 0/1, range). The methylated sequence 110b is synthesized by the methylation sequencer 102 using the patient sample 108 (e.g., blood plasma, tissue). The predicted DoR label 128 is generated by an analysis operation 124 using patient data 120.

The training system 122 is then configured to train the AI model 106 using the received methylated sequence 110b and the predicted DoR label 128. The resulting trained AI model 106b is subsequently used by the analysis and predictor system 104 to predict the DoR outcome 112b.

In FIG. 2C, the training system 122 is configured to receive (i) a methylated sequence 110c for a plurality of genes, including at least 5 of BNIP3, CES2, IGF1R, ISG15, ITGB4, KCNH2, ONECUT2, PROKR2, RUNX1, SFN, SLC22A3, SLC38A5, SMARCA2, SOX8, SST, and TACC3, and (ii) a predicted Progression-Free Survival (PFS) label 130 (e.g., 0/1, range). The methylated sequence 110c is synthesized by the methylation sequencer 102 using the patient sample 108 (e.g., blood plasma, tissue). The predicted PFS label 130 is generated by an analysis operation 124 using patient data 120.

The training system 122 is then configured to train the AI model 106 using the received methylated sequence 110c and the predicted PFS label 130. The resulting trained AI model 106c is subsequently used by the analysis and predictor system 104 to predict the PFS outcome 112c.

In FIG. 2D, the training system 122 is configured to receive (i) a methylated sequence 110d for a plurality of genes, including at least 5 of BNIP3, CES2, CHFR, CXCL5, GSTM2, IGF1R, ISG15, ITGB4, KCNH2, MUC4, ONECUT2, PROKR2, RUNX1, SFN, SLC22A3, SLC38A5, SMARCA2, SOX8, SST, and TACC3, and (ii) a predicted Time-To-Progression (TTP) label 132 (e.g., 0/1, range). The methylated sequence 110d is synthesized by the methylation sequencer 102 using the patient sample 108 (e.g., blood plasma, tissue). The predicted TTP label 132 is generated by an analysis operation 124 using patient data 120.

The training system 122 is then configured to train the AI model 106 using the received methylated sequence 110d and the predicted TTP label 132. The resulting trained AI model 106d is subsequently used by the analysis and predictor system 104 to predict the TTP outcome 112d.

Machine Learning. In addition to the artificial intelligence and/or machine learning features described above, the analysis and predictor system 104 can be implemented using one or more artificial intelligence and/or machine learning operations. The term “artificial intelligence” can include any technique that enables one or more computing devices or computing systems (i.e., a machine) to mimic human intelligence. Artificial intelligence (AI) includes but is not limited to knowledge bases, machine learning, representation learning, and deep learning. The term “machine learning” is defined herein to be a subset of AI that enables a machine to acquire knowledge by extracting patterns from raw data. Machine learning techniques include, but are not limited to, logistic regression, support vector machines (SVMs), decision trees, Naïve Bayes classifiers, and artificial neural networks. The term “representation learning” is defined herein to be a subset of machine learning that enables a machine to automatically discover representations needed for feature detection, prediction, or classification from raw data. Representation learning techniques include, but are not limited to, autoencoders and embeddings. The term “deep learning” is defined herein to be a subset of machine learning that enables a machine to automatically discover representations needed for feature detection, prediction, classification, etc., using layers of processing. Deep learning techniques include but are not limited to artificial neural networks or multilayer perceptron (MLP).

An artificial neural network (ANN) is a computing system including a plurality of interconnected neurons (e.g., also referred to as “nodes”). This disclosure contemplates that the nodes can be implemented using a computing device (e.g., a processing unit and memory as described herein). The nodes can be arranged in a plurality of layers, such as an input layer, an output layer, and optionally one or more hidden layers with different activation functions. An ANN having hidden layers can be referred to as a deep neural network or multilayer perceptron (MLP). Each node is connected to one or more other nodes in the ANN. For example, each layer is made of a plurality of nodes, where each node is connected to all nodes in the previous layer. The nodes in a given layer are not interconnected with one another, i.e., the nodes in a given layer function independently of one another. As used herein, nodes in the input layer receive data from outside of the ANN, nodes in the hidden layer(s) modify the data between the input and output layers, and nodes in the output layer provide the results. Each node is configured to receive an input, implement an activation function (e.g., binary step, linear, sigmoid, tanh, or rectified linear unit (ReLU) function), and provide an output in accordance with the activation function. Additionally, each node is associated with a respective weight. ANNs are trained with a dataset to maximize or minimize an objective function. In some implementations, the objective function is a cost function, which is a measure of the ANN's performance (e.g., error such as L1 or L2 loss) during training, and the training algorithm tunes the node weights and/or bias to minimize the cost function. This disclosure contemplates that any algorithm that finds the maximum or minimum of the objective function can be used for training the ANN. Training algorithms for ANNs include but are not limited to backpropagation. It should be understood that an artificial neural network is provided only as an example machine learning model. This disclosure contemplates that the machine learning model can be any supervised learning model, semi-supervised learning model, or unsupervised learning model. Optionally, the machine learning model is a deep learning model. Machine learning models are known in the art and are therefore not described in further detail herein.

A convolutional neural network (CNN) is a type of deep neural network that has been applied, for example, to image analysis applications. Unlike traditional neural networks, each layer in a CNN has a plurality of nodes arranged in three dimensions (width, height, depth). CNNs can include different types of layers, e.g., convolutional, pooling, and fully-connected (also referred to herein as “dense”) layers. A convolutional layer includes a set of filters and performs the bulk of the computations. A pooling layer is optionally inserted between convolutional layers to reduce the computational power and/or control overfitting (e.g., by downsampling). A fully-connected layer includes neurons, where each neuron is connected to all of the neurons in the previous layer. The layers are stacked similarly to traditional neural networks. GCNNs are CNNs that have been adapted to work on structured datasets such as graphs.

Other Supervised Learning Models. A logistic regression (LR) classifier is a supervised classification model that uses the logistic function to predict the probability of a target, which can be used for classification. LR classifiers are trained with a data set (also referred to herein as a “dataset”) to maximize or minimize an objective function, for example, a measure of the LR classifier's performance (e.g., an error such as L1 or L2 loss), during training. This disclosure contemplates that any algorithm that finds the minimum of the cost function can be used. LR classifiers are known in the art and are therefore not described in further detail herein.

A Naïve Bayes' (NB) classifier is a supervised classification model that is based on Bayes' Theorem, which assumes independence among features (i.e., the presence of one feature in a class is unrelated to the presence of any other features). NB classifiers are trained with a data set by computing the conditional probability distribution of each feature given a label and applying Bayes' Theorem to compute the conditional probability distribution of a label given an observation. NB classifiers are known in the art and are therefore not described in further detail herein.

A k-NN classifier is an unsupervised classification model that classifies new data points based on similarity measures (e.g., distance functions). The k-NN classifiers are trained with a data set (also referred to herein as a “dataset”) to maximize or minimize a measure of the k-NN classifier's performance during training. This disclosure contemplates any algorithm that finds the maximum or minimum. The k-NN classifiers are known in the art and are therefore not described in further detail herein.

A majority voting ensemble is a meta-classifier that combines a plurality of machine learning classifiers for classification via majority voting. In other words, the majority voting ensemble's final prediction (e.g., class label) is the one predicted most frequently by the member classification models. The majority voting ensembles are known in the art and are therefore not described in further detail herein.

### Example Sequence Extraction

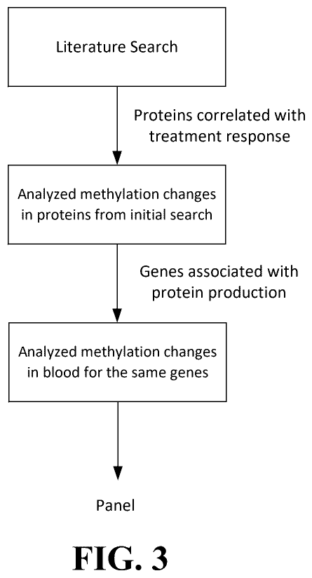

FIG. 3 shows an example sequence extraction operation configured to extract a methylated DNA or RNA sequence described in relation to FIGS. 1-2 for used in a panel. The operation was employed in a study to generate the panel described herein. The study performed a literature search to identify all proteins that may play a role in treatment response. The study then reviewed the methylation changes in those in those genes that produce those proteins to determine whether the methylation changes in those in those genes have an impact on a treatment outcome. In particular, the study evaluated 99 genes and 99 proteins. The study identified the genes associated with the protein production and tested the signature in the tissue in the TCGA database. The study observed that the those gene with the methylation changes may be identified in tissue sample, and then replicated the analysis for cfDNA in blood.

### Example Method

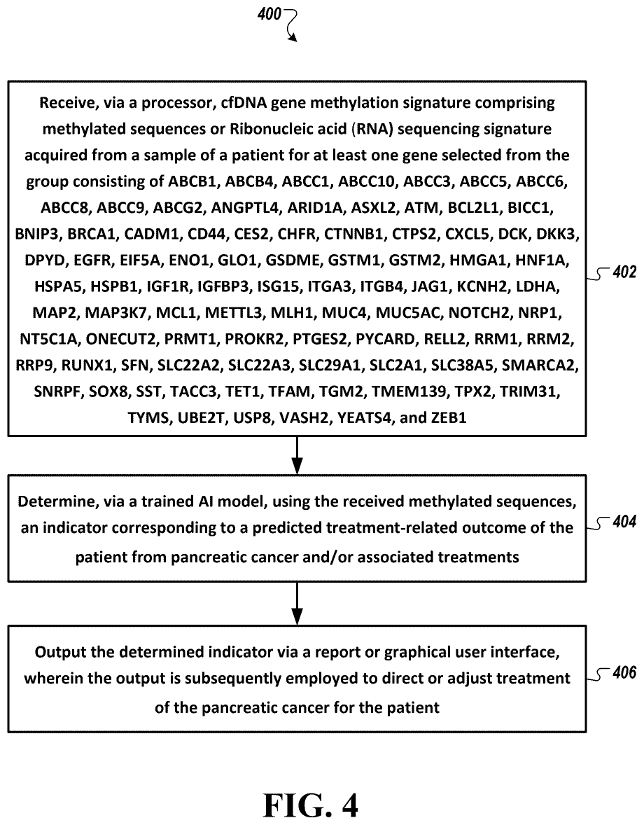

FIG. 4 shows an example operation flow 400 for the exemplary system, in accordance with an illustrative embodiment. The method 400 includes receiving (402), via a processor, a methylation signature comprising methylated nucleic acid sequences (e.g., DNA, cfDNA, RNA) or RNA sequencing signature acquired from a sample of a patient for at least one gene selected from the group consisting of ABCB1, ABCB4, ABCC1, ABCC10, ABCC3, ABCC5, ABCC6, ABCC8, ABCC9, ABCG2, ANGPTL4, ARID1A, ASXL2, ATM, BCL2L1, BICC1, BNIP3, BRCA1, CADM1, CD44, CES2, CHFR, CTNNB1, CTPS2, CXCL5, DCK, DKK3, DPYD, EGFR, EIF5A, ENO1, GLO1, GSDME, GSTM1, GSTM2, HMGA1, HNF1A, HSPA5, HSPB1, IGF1R, IGFBP3, ISG15, ITGA3, ITGB4, JAG1, KCNH2, LDHA, MAP2, MAP3K7, MCL1, METTL3, MLH1, MUC4, MUC5AC, NOTCH2, NRP1, NT5C1A, ONECUT2, PRMT1, PROKR2, PTGES2, PYCARD, RELL2, RRM1, RRM2, RRP9, RUNX1, SFN, SLC22A2, SLC22A3, SLC29A1, SLC2A1, SLC38A5, SMARCA2, SNRPF, SOX8, SST, TACC3, TET1, TFAM, TGM2, TMEM139, TPX2, TRIM31, TYMS, UBE2T, USP8, VASH2, YEATS4, and ZEB1.

Method 400 includes determining (404), via a trained AI model, using the received methylated sequences or RNA sequences, an indicator corresponding to a predicted treatment-related outcome (e.g., overall survival, duration of response, progress-free survival, time-to-progression) of the patient from pancreatic cancer and/or associated treatments.

Method 400 includes outputting (406) the determined indicator via a report or graphical user interface, wherein the output is subsequently employed to direct or adjust treatment of the pancreatic cancer for the patient.

In some embodiments, the trained AI model was trained using methylated sequences or RNA sequences for a plurality of genes, including at least 5 of BNIP3, CES2, CHFR, CXCL5, GSTM2, ITGB4, MUC4, MUC5AC, ONECUT2, PRMT1, RUNX1, SFN, SLC22A3, SOX8, TACC3, KCNH2, PROKR2, IGF1R, TMEM139, where the cfDNA gene signature is stratified for a patient population having a high risk group label and a lower risk group label for overall survival (OS).

In some embodiments, the trained AI model was trained using methylated sequences or RNA sequences for a plurality of genes, including at least 5 of BNIP3, CES2, CHFR, CXCL5, GSTM2, ITGB4, MUC4, ONECUT2, PRMT1, RUNX1, SFN, SLC22A3, SOX8, TACC3, IGF1R, KCNH2, MUC5AC, SST, and TMEM139, where the cfDNA gene signature is stratified for a patient population having a high risk group label and a lower risk group label for duration of response (DoR).

In some embodiments, the trained AI model was trained using methylated sequences or RNA sequences for a plurality of genes, including at least 5 of BNIP3, CES2, IGF1R, ISG15, ITGB4, KCNH2, ONECUT2, PROKR2, RUNX1, SFN, SLC22A3, SLC38A5, SMARCA2, SOX8, SST, and TACC3, where the cfDNA gene signature is stratified for a patient population having a high risk group label and a lower risk group label for progression-free survival (PFS).

In some embodiments, the trained AI model was trained using methylated sequences or RNA sequences for a plurality of genes, including at least 5 of BNIP3, CES2, CHFR, CXCL5, GSTM2, IGF1R, ISG15, ITGB4, KCNH2, MUC4, ONECUT2, PROKR2, RUNX1, SFN, SLC22A3, SLC38A5, SMARCA2, SOX8, SST, and TACC3, where the cfDNA gene signature is stratified for a patient population having a high risk group label and a lower risk group label for time-to-progression (TTP).

### Example Cell-Free DNA (cfDNA) Epigenetic Panel and Associated Analysis

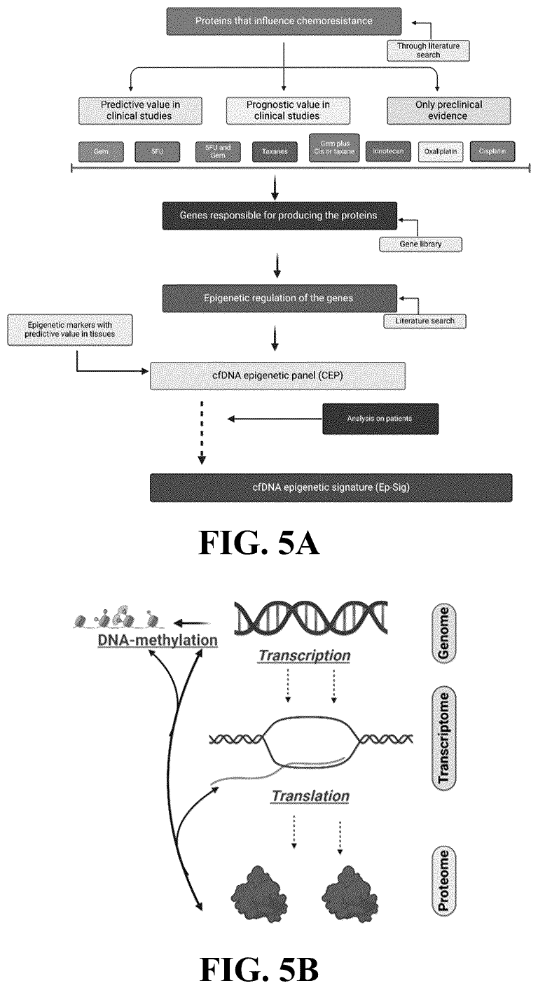

FIG. 5A shows an example methodology for creating a cfDNA epigenetic panel with all possible genes, portions of which the trained AI model is trained on to predict the treatment-related outcome for a patient. FIG. 5B shows an example relationship between methylation changes and protein expression. To identify chemoresistance without profiling patients for all drugs used in pancreatic ductal adenocarcinoma (PDA) management (e.g., Gem, NP, Iri, Ox, Cis, Nal-Iri, 5FU), a cfDNA epigenetic panel (CEP) is developed centered around DNA-methylation changes (see FIGS. 5A-5B) in the genes responsible for the expression of proteins that have association with resistance to chemotherapy drugs utilized in PDA management.

Table 1 shows an example curated cfDNA epigenetic panel for chemoresistance (with size n=99 genes).

TABLE 1
ABCB1 ABCB4 ABCC1 ABCC10 ABCC11 ABCC3 ABCC4 ABCC5 ABCC6 ABCC8 ABCC9
ABCG2 ANGPTL4 ARID1A ASXL2 ATM BCL2L1 BICC1 BNIP3 BRCA1 CADM1 CCDC85A CD44
CES2 CHFR CPT1B CTNNB1 CTPS2 CXCL5 DCK DDX3X DHX38 DKK3 DPYD EGFR EIF5A
ENO1 GLO1 GSDME GSTM1 GSTM2 HMGA1 HNF1A HSPA5 HSPB1 IGF1R IGFBP3 ISG15
ITGA3 ITGB4 JAG1 KCNH2 LDHA MAP2 MAP3K7 MCL1 METTL3 MLH1 MUC4 MUC5AC
MUTYH NOTCH2 NRP1 NT5C1A ONECUT2 PRMT1 PROKR2 PTGES2 PYCARD RELL2 RRM1
RRM2 RRP9 RUNX1 SFN SLC22A2 SLC22A3 SLC29A1 SLC2A1 SLC38A5 SLFN11 SMARCA2
SNRPF SOX8 SST TACC3 TET1 TFAM TFAP2E TGM2 TMEM139 TPX2 TRIM31 TYMS UBE2T
USP8 VASH2 YEATS4 ZEB1

Enzymatic Methylation Sequencing. cfDNA should be isolated from plasma samples for methylation sequencing. In some embodiments, the sequencing is performed using an enzymatic methylation sequencing (EM-seq) technique, where cfDNA is fragmented to a desired size range (e.g., 300 base pairs), followed by end-repair and addition of deoxyadenosine (dA) overhangs. Adapter sequences compatible with EM-seq are then ligated to the processed DNA fragments. A first enzymatic treatment (e.g., addition of TET2 and oxidation enhancer) may be applied to protect methylated cytosines (e.g., 5-methylcytosine and 5-hydroxymethylcytosine) from deamination. Subsequently, a second enzymatic treatment (e.g., addition of APOBEC enzyme) may be used to convert unmethylated cytosines to uracils (C to U). The resulting DNA library may be amplified via polymerase chain reaction (PCR), indexed (e.g., with primers), pooled, and subjected to high-throughput sequencing using a suitable sequencing platform (e.g., Illumina sequencer).

Correlation between CEP and Survival Outcomes Endpoints. In some embodiments, epigenetic profiles derived from a curated panel are evaluated in relation to survival outcomes, including time to first progression (TTP), progression-free survival (PFS), overall survival (OS), and duration of response (DOR). These analyses (e.g., univariate, multivariate) may be conducted across a population and within defined clinical subgroups. Table 2 shows the definition of survival end-points/outcomes.

TABLE 2

| Survival | |
|---|---|
| endpoint/ | |
| outcome | Definition |
| Overall | The interval from the date of diagnosis |
| survival | to the date of death or the last patient |
| (OS) | encounter, if still alive. |
| Duration of | The duration from the date of the initial |
| response (DoR) | administration of first-line chemotherapy |
| | to the date of death or the last patient |
| | encounter, if still alive. |
| Progression- | Duration from the date of diagnosis to the |
| free survival | date of first progression, death, or last |
| (PFS) | encounter, in cases where the patient did |
| | not experience progression at the time of |
| | data collection. |
| Time to | Duration from the date of the first dose of |
| progession | chemotherapy to the date of first progression, |
| (TTP) | death, or last encounter, again, if the patient |
| | did not progress at the time of data collection. |

In univariate analysis, methylation levels for each gene may be dichotomized into distinct groups (e.g., high versus low methylation) to assess associations with survival outcomes using statistical methods (e.g., log-rank testing).

In multivariate analysis, multivariate predictive modeling may be performed using regression techniques (e.g., Cox regression model with backward model selection method) with variable selection strategies to identify epigenetic and clinical contributors. Based on model-derived risk scores, subjects (e.g., patients) may be stratified into prognostic groups, and survival distributions may be visualized using Kaplan-Meier plots with corresponding statistical comparisons.

In some embodiments, epigenetic markers are mapped to cell-free DNA (cfDNA). Genes lacking consistent methylation signals across samples may be excluded from further analysis. For genes with multiple methylation sites, methylation levels may be averaged across those sites to generate a representative value. Genes may be ranked according to average methylation levels, and those below a defined threshold (e.g., 5%) may be filtered out. Sample ordering may be harmonized across molecular and clinical datasets to enable integrated analysis.

### Experimental Results and Additional Examples

A study was conducted to develop and evaluate an experimental system and method comprising (i) a methylation sequencer configured to synthesize a cfDNA methylation signature from a patient sample (e.g., blood plasma, tissue) and (ii) an analysis and predictor system having a trained AI model configured to predict the treatment-related outcome, e.g., using indicator(s) (e.g., OS, DoR, PFS, TTP), based on the cfDNA methylation signature, as described in relation to FIGS. 1-4.

### Experiment Procedure

The study recruited 71 PDA patients, only 51 of whom were eligible to form a study cohort (SC) for the experiment evaluating the experimental system and method. The study also developed a cfDNA epigenetic panel based on the methodology described in relation to FIG. 5A.

Enzymatic methylation sequencing. Then, the study isolated cfDNA from the plasma of PDA patients for methylation sequencing. Methylation sequencing in the study employed an Enzymatic Methylation Sequencing (EM-Seq) technique using the New England Biolaboratories EM-Seq kit. During the sequencing, 10-200 ng of cfDNA was sheared to −300 bp fragments using the Covaris S2 ultrasonicator. The fragmented DNA was then end-repaired and dA-tailed, followed by EM-seq adaptor ligation. The first enzyme, TET2, and an oxidation enhancer were added to protect 5mC/5hmC from deamination, and then the second enzyme, APOBEC, was added to deaminate cytosines to uracils (C to U). The library was prepared by PCR amplification, and the PCR products were labeled with index primers. Finally, the libraries were pooled and sequenced using an Illumina sequencer.

Statistical Analysis. The study correlated the CEP to survival endpoints: time to first progression (TTP), progression-free survival (PFS), overall survival (OS), and total duration of response (DOR) for the entire study cohort (SC) and the palliative treatment (PT) subgroup.

In univariate analyses of the experiment, the study dichotomized methylation levels of all samples for each gene into high methylation (‘High’) or low methylation (‘Low’) groups. Log-rank test p-values were obtained to test the difference between high and low methylation groups for each gene.

In the multivariate analyses of the experiment, the study built multivariate predictive models using the Cox regression model with the backward model selection method. Significant genes in the final model were identified, and critical clinical variables were adjusted. Then, the study divided patients into high-risk and low-risk groups based on the risk scores of the final model. A Kaplan-Meier curve was plotted along with the log-rank test to test the difference between the two risk groups.

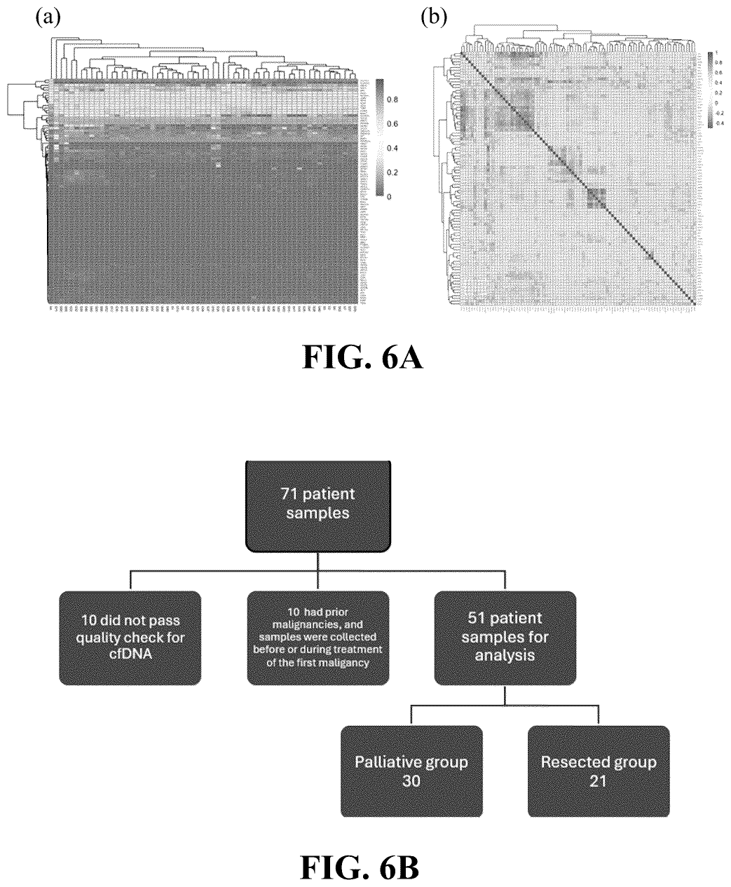

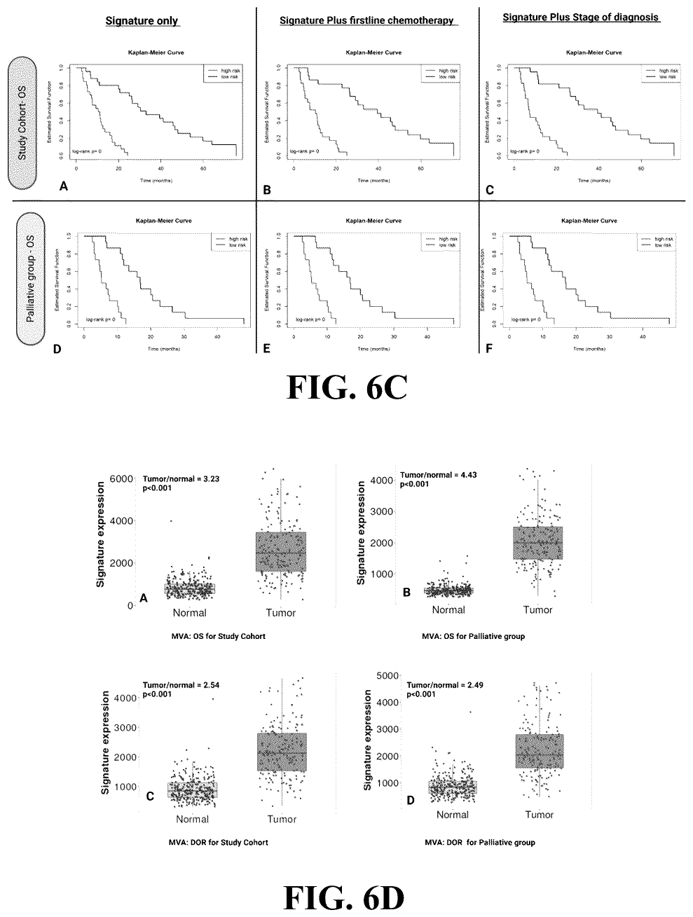

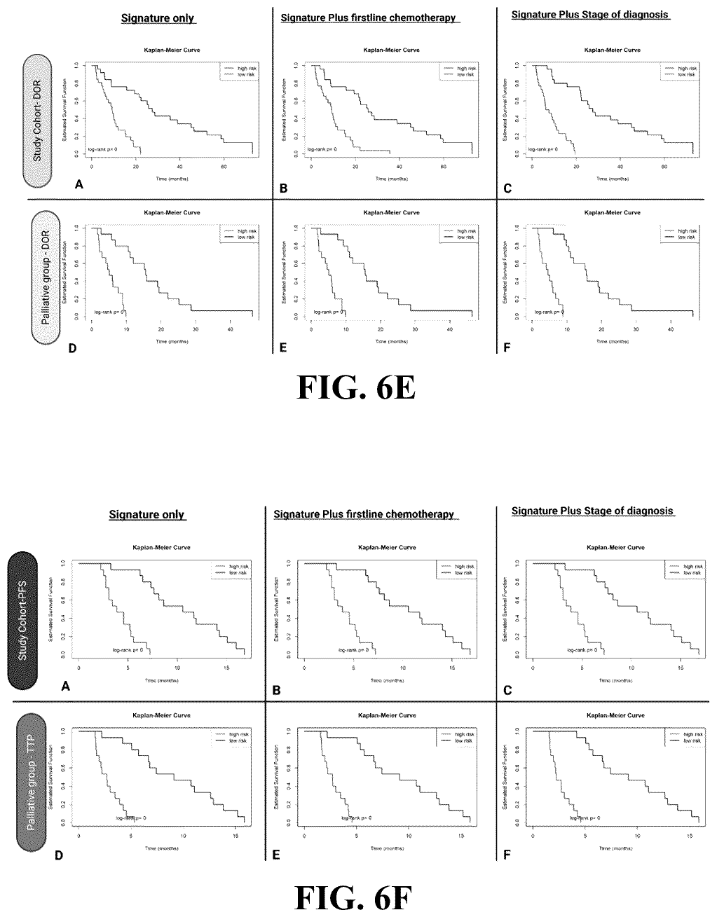

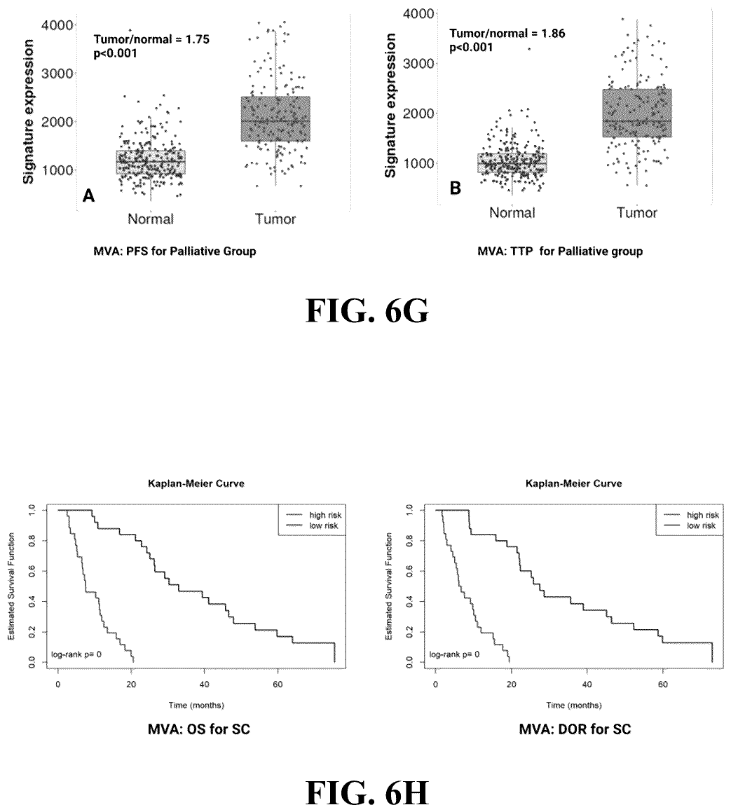

The study located the 99 genes in CEP in the cfDNA (see Table 1). However, 10 genes (e.g., ABCC11, ABCC4, CCDC85A, CPT1B, DDX3X, DHX38, MUTYH, PYCARD, SLFN11, TFAP2E) did not have positive methylation across all samples. FIG. 6A shows methylation levels in the patient population (71 patients) of the experiment. Consistent methylation levels were observed across multiple locations within genes from the heatmap. Therefore, methylation levels were averaged across locations within each gene (see FIG. 6A, subpanels (a)-(b)). The average methylation levels across all 61 samples were ranked from highest to lowest. Genes with an average methylation level of less than 5% were filtered out. Samples were aligned in the same order for clinic and methylation data.

### Experiment Results

The study collected samples (e.g., plasma) from 71 patients before the first dose of chemotherapy or during the early chemotherapy period. FIG. 6B shows a breakdown of available samples for the analysis in the experiment. As shown, the study only analyzed the cohort of 51 patients (out of 71), which comprised two subgroups: a palliative treatment (PT) subgroup of 30 and a resected (Rs) group of 21. Table 3 shows the baseline characteristics of the study cohort of 51 patients.

TABLE 3

| Characteristics | Distribution |
|---|---|
| Age of diagnosis | 65 years (range: 34-80) |
| Race | Caucasian - 86% |
| | African-American - 12% |
| | Others - 2% |
| Smoking history | 65% |
| Alcoholic history | 57% |
| CA19-9 | Median 311 ng/mL |
| | (range: undetectable - 41, 258) |
| Tumor primary | Head - 38 (74%) |
| sites n(%) | Body - 6 (12%) |
| | Tail - 5 (10%) |
| | Overlapping - 2 (4%) |
| Stage of diagnosis | Early-stage (I/II) - 21 (43%) |
| | 9 had UpS |
| | 13 had NAT, but 3 progressed, and 10 had |
| | surgery |
| | 3 progressed - 2 FFX and 1 Gem-NP |
| | Surgery - 5 FFX. 1 FOLFOX, 1 had CRT |
| | followed by FOLFOX, 1-Gem-NP, 2 FXX |
| | switched to Gem-NP |
| | Advanced stage - 30 (67%) |
| | Locally advanced - 15 (2 responded well |
| | to proceed to surgery, got FFX) |
| | Metastatic - 14 |
| | 2 patients in LA proceeded to have surgery |
| | 3 from stage I/II proceeded to have palliative |
| | treatment |
| Treatment groups | Palliative treatment - 30 |
| | Resected - 21 (UpS - 9, NAT- 12) |
| First-line | Palliative - 30 |
| chemotherapy | FFX - 12 |
| for analysis | G-NP - 16 |
| | FOLFOX - 1 |
| | Gem-only - 1 |
| | Resected |
| | UpS - 9 |
| | Adjuvant |
| | FFX - 5 |
| | GA-2 |
| | Gem only 1 |
| | Gem/cap - 1 |
| | NAT = 2 LA and 10 BR/R - 7 FFX. 1 |
| | FOLFOX, 1 had CRT followed by |
| | FOLFOX, 1-GA, 2 FFX switched to Gem-NP |
| | For analysis |
| | FFX - 26 |
| | Gem-NP-19 |
| | Other - 6 (3 FOLFOX, 1 Gem-only, 1-Gem/cap) |
| Analysis of | 1. FFX and Gem-NP = 14 |
| the treatment | 2. FFX at some point = 27 |
| received | 3. Gem-NP at some point = 37 |
| | Palliative group |
| | 1. FFX and GA = 6 |
| | 2. FFX at some point = 12 |
| | 3. G-NP at some point = 24 |

Note:
FFX = FOLFIRINOX, Gem = gemcitabine, NP = nab-paclitaxel, cap = capecitabine.

The results of the study were categorized into four survival endpoints/outcomes, including OS, DoR, PFS, and TTP, as defined per Table 2.

The results for the entire study cohort (SC, n=51) and the palliative treatment subgroup (PT, 30/51) were presented for OS and DOR. The PFS and TTP outcomes were reported exclusively for the PT group. Due to the limited sample size of patients who underwent resection (Rs, 21/51), an analysis of PFS and TTP in this subgroup was not feasible. Finally, the study introduced a comprehensive model integrating multiple clinical variables for OS and DOR in the SC group. This model was presented separately since most selected variables apply exclusively to SC, rather than the PT or Rs groups.

For each survival outcome/endpoint, the study presented methylation changes in univariate analysis, followed by gene signatures demonstrating significance in multivariate analysis (MVA). For MVA, the study presented the gene signature alone and then incorporated clinical variables, first with first-line chemotherapy (FLC) and subsequently with stage at diagnosis (Std). In the Rs subgroup, FLC was administered as adjuvant therapy (following upfront surgery) or neoadjuvant therapy. Furthermore, the study compared RNA expression of the identified signatures (developed in the study) between normal and malignant pancreatic tissues using publicly available datasets (TNMplot.com). Table 4 shows the durations of sample collection for the SC, PT subgroup, and Rs subgroup.

TABLE 4

| Group | Duration of sample collection |
|---|---|
| SC | Median sample collection occurred 8 days before the |
| | first dose of chemotherapy (range: 95 days before |
| | to 56 days after chemotherapy). |
| PT | Median sample collection was 9 days before the first |
| | dose of chemotherapy (range: 66 days before to 56 days |
| | after chemotherapy). |
| | One patient with a sample collected 66 days before |
| | surgery was initially diagnosed as borderline |
| | resectable (BR) and underwent upfront surgery, but was |
| | found to have metastatic disease intraoperatively. |
| | Another patient with a sample collected 56 days after |
| | chemotherapy initiation had BR disease and was also |
| | found to have metastases intraoperatively. |
| Rs | Median sample collection was 7 days before surgery |
| | (range: 95 days before to 55 days after the first |
| | chemotherapy dose). Most patients who had samples |
| | collected far before chemotherapy initiation underwent |
| | upfront surgery. |

Overall Survival (OS) Outcome. Table 5 shows a univariate analysis for OS for the study cohort (SC) and palliative treatment (PT) subgroup.

TABLE 5

| | Study cohort (SC) | | Palliative Treatment (PT) | |
|---|---|---|---|---|
| Gene | Hazard ratio | p-value | Hazard ratio | p-value |
| MUC5AC | 2.051972 | 0.013687 | | |
| SST | 1.920555 | 0.028503 | 2.2942795 | 0.032781 |
| SLC22A3 | 1.882253 | 0.029399 | | |
| SFN | 1.791787 | 0.049637 | | |
| ONECUT2 | 1.692023 | 0.075208 | | |
| PRMT1 | | | 2.186757 | 0.042209 |

FIG. 6C shows high-risk (H) and low-risk (L) groups for overall survival in the study cohort (SC) and palliative treatment (PT) subgroup. Table 6 shows a 15-gene signature (also referred to as a multivariate analysis (MVA) signature for OS in the SC (OS-SC)) that stratified the population into high-risk (H) and low-risk (L) groups for overall survival in the SC (see FIG. 6C, subpanels (a)-(c)).

TABLE 6

| | |
|---|---|
| MVA for | BNIP3 CES2 CHFR CXCL5 GSTM2 ITGB4 MUC4 |
| OS-SC | MUC5AC ONECUT2 PRMT1 RUNX1 SFN SLC22A3 |
| | SOX8 TACC3 |

FIG. 6D shows the diagnostic value of the 15-gene signature (see Table 6) for overall survival (OS) and duration of response (DoR) for the study cohort (SC) and palliative treatment (PT) subgroup. In FIG. 6D, subpanel (a), the 15-gene signature demonstrated more than threefold expression in tumor tissue compared to normal tissue. Table 7 shows other multivariate models (e.g., signature plus first-line chemotherapy, signature plus stage of diagnosis) for OS in the SC, besides the 15-gene signature only (see Table 6), and associated analysis values.

TABLE 7

| Models tested | OS^{m} (L vs. H) | Hazard ratio | p-value |
|---|---|---|---|
| Signature-alone | 10.75 vs. 33 | 8.7114 | 3.70628553403307e−08 |
| Signature plus first-line chemotherapy | 10.62 vs. 33 | 8.1015 | 3.01209893693866e−08 |
| (FOLFIRINOX vs. G-NP vs. other) | | | |
| Signature plus stage of diagnosis | 8.4 vs. 33 | 16.9874 | 1.69922853565652e−10 |
| (LA vs. mets vs. ES) | | | |

Note:
^{m}= months;
LA = locally advanced;
mets = metastatic;
ES = early stage (borderline resectable and resectable)

Table 8 shows a 15-gene signature (also referred to as an MVA signature for OS in the PT subgroup (OS-pall)) that stratified the population into high-risk (H) and low-risk (L) groups for overall survival in the PT subgroup (see FIG. 6C, subpanels (d)-(f)).

TABLE 8

| | |
|---|---|
| MVA for OS-pall | CES2 CHFR CXCL5 GSTM2 ITGB4 ONECUT2 |
| | PRMT1 RUNX1 SFN SLC22A3 TACC3 KCNH2 |
| | PROKR2 IGF1R TMEM139 |
| Exclusive to OS-pall | IGF1R TMEM139 |
| Differ from OS-SC | KCNH2 PROKR2 |

In FIG. 6D, subpanel (b), the 15-gene signature demonstrated more than fourfold expression in tumor tissue compared to normal tissue. Table 9 shows other multivariate models (e.g., signature plus first-line chemotherapy, signature plus stage of diagnosis) for OS in the PT subgroup, besides the 15-gene signature only (see Table 8), and associated analysis values.

TABLE 9

| Models tested | OS^{m} | Hazard ratio | p-value |
|---|---|---|---|
| Signature-alone | 5.3 vs. 16.83 | 9.257 | 3.34942588098297e−06 |
| Signature plus first-line chemotherapy | 5.3 vs. 16.83 | 9.257 | 3.34942588098297e−06 |
| Signature plus stage of diagnosis | 5.3 vs. 16.83 | 8.0532 | 5.54208601843964e−06 |
| (LA vs. mets vs. ES) | | | |

Note:
^{m}= months;
LA = locally advanced;
mets = metastatic;
ES = early stage (borderline resectable and resectable)

Duration of Response (DoR) Outcome. Table 10 shows a univariate analysis for DoR for the study cohort (SC) and palliative treatment (PT) subgroup.

TABLE 10

| | Study cohort (SC) | | Palliative treatment (PT) | |
|---|---|---|---|---|
| Gene | Hazard ratio | p-value | Hazard ratio | p-value |
| MUC5AC | 2.1129113 | 0.01295949 | | |
| SST | 2.1129113 | 0.01295949 | | |
| SLC22A3 | 1.7383476 | 0.05715110 | 2.1213928 | 0.05271214 |
| SFN | 1.6288049 | 0.09733143 | | |
| ONECUT2 | 1.7706915 | 0.05323394 | | |
| PRMT1 | | | 2.1483804 | 0.04841334 |

FIG. 6E shows high-risk (H) and low-risk (L) groups for DoR in the study cohort (SC) and palliative treatment (PT) subgroup. Table 11 shows a 15-gene signature (an MVA signature for DoR in the SC (DoR-SC)) and a 16-gene signature (an MVA signature for DoR in the PT subgroup (DoR-pall)) that stratified the population into high-risk (H) and low-risk (L) groups for DoR in the SC and PT subgroup, respectively (see FIG. 6E, subpanels (a)-(f)).

TABLE 11

| | |
|---|---|
| MVA for | BNIP3 CES2 CHFR CXCL5 GSTM2 ITGB4 |
| DoR-SC | MUC4 ONECUT2 PRMT1 RUNX1 SFN |
| | SLC22A3 SOX8 TACC3 IGF1R |
| MVA for | BNIP3 CHFR CXCL5 IGF1R ITGB4 KCNH2 |
| DoR-pall | MUC4 MUC5AC ONECUT2 PRMT1 SLC22A2 |
| | SLC22A3 SOX8 SST TACC3 TMEM139 |

In FIG. 6D, subpanels (c) and (d), the 15-gene and 16-gene signatures demonstrated more than twofold expression in tumor tissue compared to normal tissue. Table 12 shows other multivariate models (e.g., signature plus first-line chemotherapy, signature plus stage of diagnosis) for DoR in the SC and PT subgroup, besides the signatures only (see Table 11), and associated analysis values.

TABLE 12

| | Study cohort (n = 51) | | Palliative (n = 30) | |
|---|---|---|---|---|
| Models tested | DOR^{m} | HR^{p} | DOR^{m} | HR^{p} |
| Signature-alone | 9.28 vs. 27.5 | 6.4254 | 5.07 vs. 15.57 | 11.706 |
| Signature plus first-line chemotherapy | 9.28 vs. 28.3 | 4.7901 | 5.07 vs. 15.57 | 14.9604 |
| Signature plus stage of diagnosis | 6.88 vs. 27.5 | 10.8224 | 4.57 vs. 15.57 | 45.5023 |
| (LA vs. mets vs. ES) | | | | |

Note:
^{m}= months; ^{p}= p-value < 0.01; HR = hazard ratio; LA = locally advanced; mets = metastatic; ES = early stage (borderline resectable and resectable).

Progression-Free Survival (PFS) and Time to First Progression (TTP) Outcomes. Table 13 shows a univariate analysis for PFS and TTP for the palliative treatment (PT) subgroup.

TABLE 13

| | PFS | | TTP | |
|---|---|---|---|---|
| Gene | Hazard ratio | p-value | Hazard ratio | p-value |
| ITGB4 | 2.1621019 | 0.03970437 | 2.1993440 | 0.03724936 |
| TMEM139 | 2.2343839 | 0.04838210 | 2.4525543 | 2.4525543 |

FIG. 6F shows high-risk (H) and low-risk (L) groups for PFS (see subpanels (a)-(c)) and TTP (see subpanels (d)-(f)) in the PT subgroup. Table 14 shows a 16-gene signature (an MVA signature for PFS in the PT (PFS-pall)) and a 20-gene signature (an MVA signature for TTP in the PT subgroup (TTP-pall)) that stratified the population into high-risk (H) and low-risk (L) groups for PFS and TTP in the PT subgroup.

TABLE 14

| | |
|---|---|
| MVA for | BNIP3 CES2 IGF1R ISG15 ITGB4 KCNH2 |
| PFS-pall | ONECUT2 PROKR2 RUNX1 SFN SLC22A3 |
| | SLC38A5 SMARCA2 SOX8 SST TACC3 |
| MVA for | BNIP3 CES2 CHFR CXCL5 GSTM2 IGF1R |
| TTP-pall | ISG15 ITGB4 KCNH2 MUC4 ONECUT2 |
| | PROKR2 RUNX1 SFN SLC22A3 SLC38A5 |
| | SMARCA2 SOX8 SST TACC3 |

FIG. 6G shows the diagnostic value of the 16-gene signature and 20-gene signature (see Table 14) for progression-free survival (PFS) and time to first progression (TTP) for the palliative treatment (PT) subgroup. In FIG. 6G, subpanels (a) and (b), the 16-gene signature and 20-gene signature demonstrated more than twofold expression in tumor tissue compared to normal tissue. Table 15 shows other multivariate models (e.g., signature plus first-line chemotherapy, signature plus stage of diagnosis) for PFS and TTP in the PT subgroup, besides the signatures only (see Table 14), and associated analysis values.

TABLE 15

| | PFS^{m} | HR^{p} | TTP^{m} | HR^{p} |
|---|---|---|---|---|
| Signature-alone | 3.87 vs. 10.57 | 14.3931 | 2.7 vs. 9.13 | 15.1437 |
| Signature plus first-line chemotherapy | 3.87 vs. 10.57 | 14.3931 | 2.7 vs. 9.13 | 42.6622 |
| Signature plus stage of diagnosis | 3.87 vs. 10.57 | 14.3931 | 2.23 vs. 9.13 | 49.4175 |
| (LA vs. mets vs. ES) | | | | |

Note:
^{m}= months; ^{p}= p-value < 0.01; HR = hazard ratio; LA = locally advanced; mets = metastatic; ES = early stage (borderline resectable and resectable).

Comprehensive Model for OS and DoR. A comprehensive model for OS and DoR can integrate the signatures for OS and DOR with six clinic variables, including age of diagnosis, gender, first-line chemotherapy (FLC), stage of diagnosis (StD), first step in management, and treatment with radiation at any point in cancer management (see FIG. 6H). The first step in management may significantly impact OS and DOR. Table 16 shows a comprehensive model for OS and DoR in the SC and associated analysis values. FIG. 6H shows high-risk (H) and low-risk (L) groups for OS and DoR in the study cohort, using the comprehensive model that integrates various clinical variables.

TABLE 16

| | OS^{m} | HR^{p} | DOR^{m} | HR^{p} |
|---|---|---|---|---|
| Comprehensive model | 7.58 vs. 33 | 17.9543 | 6.45 vs. 27.5 | 12.0672 |

^{m}= months,
^{p}= p value < 0.01;
HR = hazard ratio

### Experimental Implementation of an AI Model

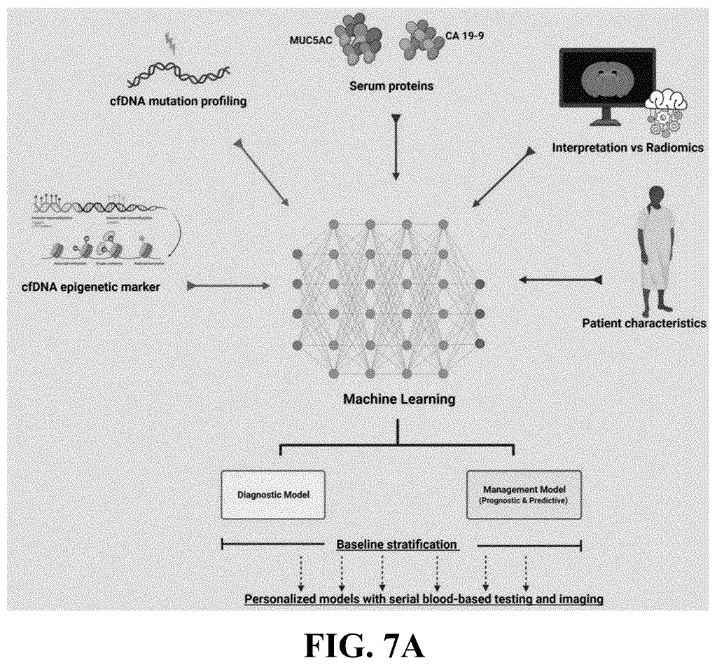

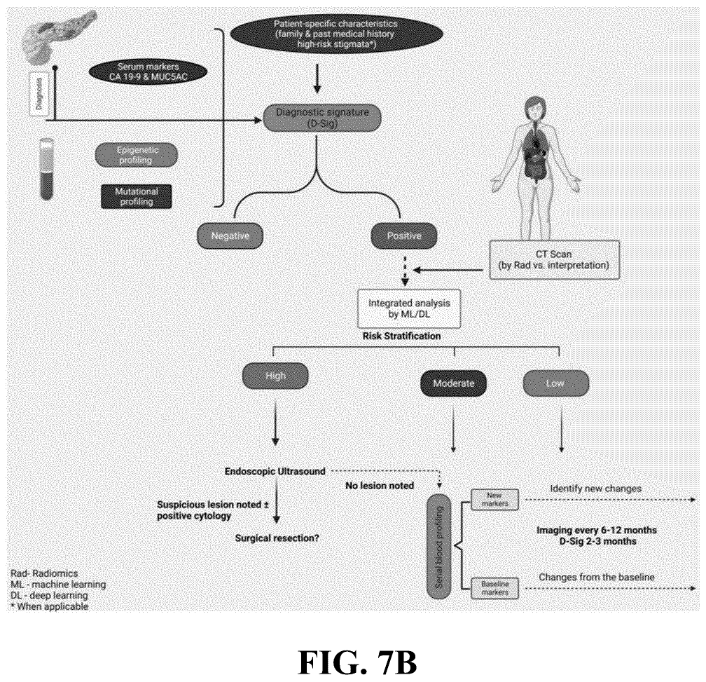

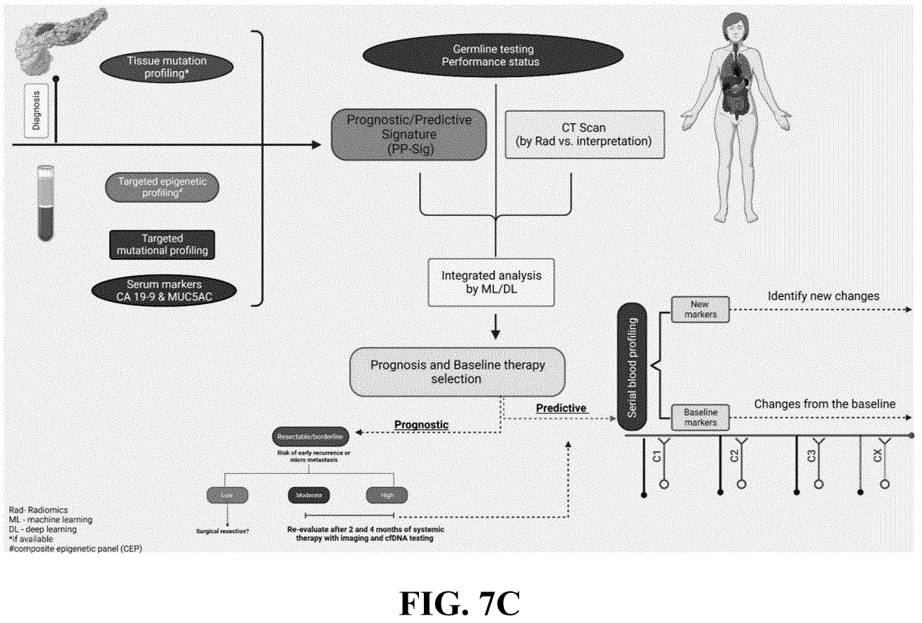

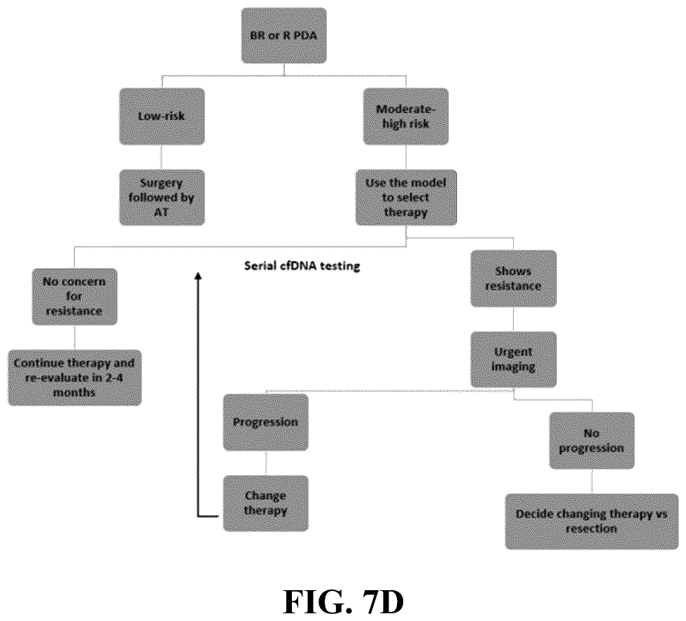

FIG. 7A shows an experimental AI model configured to combine serum proteins, an integrated cell-free DNA (cfDNA) panel, and imaging techniques to diagnose and manage pancreatic ductal adenocarcinoma (PDA).

To achieve the experimental AI model, the study first developed a PDA diagnostic model (DM) and then a PDA prognostic and predictive model (PPM). FIG. 7B shows a schema of the PDA diagnostic model (DM). As shown, the PDA diagnostic model comprises three main components: a diagnostic signature (D-Sig) (e.g., biomarkers), an imaging system (e.g., CT scan), and patient-specific characteristics. Specifically, diagnostic signature (D-Sig) (e.g., biomarkers) in the blood included serum proteins, carbohydrate antigen 19-9 (CA 19-9), circulating mucin 5AC (MUC5AC), and cell-free DNA (cfDNA) profiling (e.g., mutations and methylation changes, also known as epigenetic markers). The study used machine learning (ML) or deep learning (DL) applications (e.g., radiomics or computer vision) as the imaging system in the model to risk-stratify the patients. Patient-specific characteristics included demographics, past medical or family histories, and high-risk stigmata applicable to certain populations (e.g., Intraductal Papillary Mucinous Neoplasm (IPMN) or pancreatic cyst).

It may be uncommon for all high-risk populations (e.g., individuals with a family history of gastrointestinal/genitourinary malignancies or newly diagnosed diabetics) to have access to both medical imaging and radiomics, even when imaging is available. To address this limitation, the study developed alternative approaches to make the PDA diagnostic model (DM) feasible across these populations. Specifically, if imaging was unavailable, the PDA diagnostic model could initiate the D-Sig test first. If any component of the test (e.g., cfDNA, MM, or CA 19-9) was positive in a high-risk population, the result could be considered positive. This would then be followed by imaging, after which risk stratification would proceed according to the schema in FIG. 7B.

If radiomics were unavailable, the PDA diagnostic model could use image interpretations from radiology notes/reports, including, but not limited to, size of the lesion (if detected or in patients with IPMN or any cysts), solid vs. cystic component, cyst wall thickening, and duct dilation.

FIG. 7C shows a schema of the PDA prognostic and predictive model (PPM). As shown, the PPM comprises three main components: a prognostic/predictive signature (PP-Sig) (e.g., biomarkers), an imaging system (e.g., CT scan), and patient-specific characteristics. Specifically, prognostic/predictive signature (PP-Sig) (e.g., biomarkers) in the blood included MM, CA 19-9, and cfDNA components (mutations and EM). The study used machine learning (ML) or deep learning (DL) applications (e.g., radiomics or computer vision) as the imaging system in the model to risk-stratify the patients. Patient-specific characteristics of interest in the PPM included germline testing and performance status (PS).

The PPM was the same as the DM regarding the imaging system. If imaging and/or radiomics were unavailable, the PPM could use interpretation in radiology reports/notes with parameters including, but not limited to, size/location of the primary lesion, artery/vein involvement, and number/location of metastatic sites.

In advanced tumors, the predictive value of PPM is important. In early-stage PDA, the benefit of neoadjuvant (NAT) and an appropriate chemotherapy regimen for it is unclear [59′-63′]. FIG. 7D shows a predictive model, employed as an ML/DL application in the DM and/or PPM, configured to (i) identify, via baseline risk stratification, those who may benefit from NAT (moderate and high risk for recurrence or micro metastasis), (ii) help selecting appropriate NAT regimen, and (iii) identify resistance to therapy (and risk of disease progression) before restaging imaging so that physicians can decide on surgery or continuing/changing the systemic therapy. Similarly, serial post-operative cfDNA and serum protein testing can help develop personalized models for surveillance in patients who have had curative surgeries. In advanced or early-stage PDA, imaging is advised if PP-Sig is concerning for resistance to first-line therapy. If imaging confirms disease progression, therapy should be changed.

### CONCLUSION

As used in the specification and the appended claims, the singular forms “a,” “an” and “the” include plural referents unless the context clearly dictates otherwise. Ranges may be expressed herein as from “about” one particular value, and/or to “about” another particular value. When such a range is expressed, another implementation includes from the one particular value and/or to the other particular value. Similarly, when values are expressed as approximations, by use of the antecedent “about,” it will be understood that the particular value forms another implementation. It will be further understood that the endpoints of each of the ranges are significant both in relation to the other endpoint and independently of the other endpoint.

“Optional” or “optionally” means that the subsequently described event or circumstance may or may not occur and that the description includes instances where said event or circumstance occurs and instances where it does not.

Throughout the description and claims of this specification, the word “comprise” and variations of the word, such as “comprising” and “comprises,” means “including but not limited to,” and is not intended to exclude, for example, other additives, components, integers or steps. “Exemplary” means “an example of” and is not intended to convey an indication of a preferred or ideal implementation. “Such as” is not used in a restrictive sense but for explanatory purposes.

Disclosed are components that can be used to perform the disclosed methods and systems. These and other components are disclosed herein, and it is understood that when combinations, subsets, interactions, groups, etc. of these components are disclosed while specific reference of each various individual and collective combinations and permutation of these may not be explicitly disclosed, each is specifically contemplated and described herein, for all methods and systems. This applies to all aspects of this application, including, but not limited to, steps in disclosed methods. Thus, if there are a variety of additional steps that can be performed it is understood that each of these additional steps can be performed with any specific implementation or combination of implementations of the disclosed methods.

The following patents, applications, and publications, as listed below and throughout this document, are hereby incorporated by reference in their entirety herein.

### REFERENCE LIST #1

[1] Manne, A., et al., Abstract A014: Protein-informed gene methylation signatures to predict treatment response and outcomes in pancreatic ductal adenocarcinoma. Cancer Research, 2024. 84(17_Supplement_2): p. A014-A014.
[2] Manne, A., et al., Abstract B111: Developing prognostic signatures (ep-Sigs) through epigenetic predictive markers in pancreatic ductal adenocarcinoma (PDA)—A pilot study based on The Cancer Genome Atlas (TCGA) data. Cancer Research, 2024. 84(2_Supplement): p. B111-B111.
[3] Manne, A., et al., Developing cell-free DNA (cfDNA) epigenetic signature (ep-Sig) for early detection of malignant transformation of intraductal papillary mucinous neoplasms (IPMN). Journal of Clinical Oncology, 2024. 42(3_suppl): p. 616-616.
[4] Puram, H., et al., Abstract A013: Advanced Gene Signatures for the Diagnosis and Personalized Treatment of Pancreatic Ductal Adenocarcinoma. Cancer Research, 2024. 84(17_Supplement_2): p. A013-A013.
[5] Sherpally, D., et al., More isn't always better: Expanding and refining prognostic methylation signatures for pancreatic ductal adenocarcinoma. Journal of Clinical Oncology, 2025. 43(4_suppl): p. 769-769.
[6] Sherpally, D., et al., Predictive methylation signature for gemcitabine sensitivity in pancreatic ductal adenocarcinoma: A pathway to personalized treatment. Journal of Clinical Oncology, 2025. 43(4_suppl): p. 768-768.

### REFERENCE LIST #2

[1′] George, B., et al., Comprehensive genomic profiling (CGP) utilizing cell-free DNA (cfDNA) in patients (pts) with pancreatic ductal adenocarcinoma (PDAC). Journal of Clinical Oncology, 2021. 39(3_suppl): p. 421-421.
[2′] George, B., et al., Correlation between comprehensive genomic profiling (CGP) utilizing tissue-based testing (TCGP) and cell-free DNA (cfDNA) in patients (pts) with pancreatic ductal adenocarcinoma (PDAC). Journal of Clinical Oncology, 2021. 39(3_suppl): p. 422-422.
[3′] Botrus, G., et al., Serial cell-free DNA (cfDNA) sampling in advanced pancreatic ductal adenocarcinoma (PDAC) patients may predict therapeutic outcome. Journal of Clinical Oncology, 2021. 39(3_suppl): p. 423-423.
[4′] Natale, F., et al., Deciphering DNA methylation signatures of pancreatic cancer and pancreatitis. Clinical Epigenetics, 2019. 11(1).
[5′] Shi, Z., et al., Systematic evaluation of cancer-specific genetic risk score for 11 types of cancer in The Cancer Genome Atlas and Electronic Medical Records and Genomics cohorts. Cancer Medicine, 2019. 8(6): p. 3196-3205.
[6′] Wolpin, B. M., et al., Genome-wide association study identifies multiple susceptibility loci for pancreatic cancer. Nature genetics, 2014. 46(9): p. 994-1000.
[7′] Petersen, G. M., et al., A genome-wide association study identifies pancreatic cancer susceptibility loci on chromosomes 13q22.1, 1q32.1 and 5p15.33. Nature genetics, 2010. 42(3): p. 224-228.
[8′] Childs, E. J., et al., Common variation at 2p13.3, 3q29, 7p13 and 17q25.1 associated with susceptibility to pancreatic cancer. Nature genetics, 2015. 47(8): p. 911-916.
[9′] Sheel, A., et al., Is Cell-Free DNA Testing in Pancreatic Ductal Adenocarcinoma Ready for Prime Time?Cancers, 2022. 14(14): p. 3453.
[10′] Bara, A. W., A. Braszewska, and J. Kwasniewska, DNA Methylation—An Epigenetic Mark in Mutagen-Treated Brachypodium distachyon Cells. Plants, 2021. 10(7): p. 1408.
[11′] Fujimoto, Y., et al., Combination of CA19-9 and Blood Free-Circulating Methylated RUNX3 May Be Useful to Diagnose Stage I Pancreatic Cancer. Oncology, 2021. 99(4): p. 234-239.
[12′] Henriksen, S. D., et al., Cell-free DNA promoter hypermethylation in plasma as a diagnostic marker for pancreatic adenocarcinoma. Clinical Epigenetics, 2016. 8(1).
[13′] Guler, G. D., et al., Detection of early stage pancreatic cancer using 5-hydroxymethylcytosine signatures in circulating cell free DNA. Nat Commun, 2020. 11(1): p. 5270.
[14′] Ying, L., et al., Methylation-based Cell-free DNA Signature for Early Detection of Pancreatic Cancer. Pancreas, 2021. 50(9): p. 1267-1273.
[15′] Manoochehri, M., et al., SST gene hypermethylation acts as a pan-cancer marker for pancreatic ductal adenocarcinoma and multiple other tumors: toward its use for blood-based diagnosis. Mol Oncol, 2020. 14(6): p. 1252-1267.
[16′] Li, X. B., et al., Non-invasive detection of pancreatic cancer by measuring DNA methylation of Basonuclin 1 and Septin 9 in plasma. Chin Med J (Engl), 2019. 132(12): p. 1504-1506.
[17′] Singh, N., et al., Clinical significance of promoter methylation status of tumor suppressor genes in circulating DNA of pancreatic cancer patients. J Cancer Res Clin Oncol, 2020. 146(4): p. 897-907.
[18′] Kandimalla, R., et al., EpiPanGI Dx: A Cell-free DNA Methylation Fingerprint for the Early Detection of Gastrointestinal Cancers. Clinical Cancer Research, 2021: p. clincanres. 1982.
[19′] Li, S., et al., Genome-Wide Analysis of Cell-Free DNA Methylation Profiling for the Early Diagnosis of Pancreatic Cancer. Frontiers in Genetics, 2020. 11.
[20′] Vrba, L., et al., Liquid biopsy, using a novel DNA methylation signature, distinguishes pancreatic adenocarcinoma from benign pancreatic disease. Clinical Epigenetics, 2022. 14(1).
[21′] Park, J. K., et al., The role of quantitative NPTX2 hypermethylation as a novel serum diagnostic marker in pancreatic cancer. Pancreas, 2012. 41(1): p. 95-101.
[22′] Park, J. W., I. H. Baek, and Y. T. Kim, Preliminary Study Analyzing the Methylated Genes in the Plasma of Patients with Pancreatic Cancer. Scandinavian Journal of Surgery, 2012. 101(1): p. 38-44.
[23′] Melson, J., et al., Commonality and differences of methylation signatures in the plasma of patients with pancreatic cancer and colorectal cancer. Int J Cancer, 2014. 134(11): p. 2656-62.
[24′] Cao, F., et al., Integrated epigenetic biomarkers in circulating cell-free DNA as a robust classifier for pancreatic cancer. Clin Epigenetics, 2020. 12(1): p. 112.
[25′] Eissa, M. A. L., et al., Promoter methylation of ADAMTS1 and BNC1 as potential biomarkers for early detection of pancreatic cancer in blood. Clin Epigenetics, 2019. 11(1): p. 59.
[26′] Shinjo, K., et al., A novel sensitive detection method for DNA methylation in circulating free DNA of pancreatic cancer. PLOS ONE, 2020. 15(6): p. e0233782.
[27′] Lehmann-Werman, R., et al., Identification of tissue-specific cell death using methylation patterns of circulating DNA. Proc Natl Acad Sci USA, 2016. 113(13): p. E1826-34.
[28′] Miller, B. F., H. M. Petrykowska, and L. Elnitski, Assessing ZNF154 methylation in patient plasma as a multicancer marker in liquid biopsies from colon, liver, ovarian and pancreatic cancer patients. Scientific Reports, 2021. 11(1).
[29′] Pedersen, K. S., et al., Leukocyte DNA Methylation Signature Differentiates Pancreatic Cancer Patients from Healthy Controls. PLoS ONE, 2011. 6(3): p. e18223.
[30′] Liggett, T., et al., Differential methylation of cell-free circulating DNA among patients with pancreatic cancer versus chronic pancreatitis. Cancer, 2010. 116(7): p. 1674-1680.
[31′] Hong, S.-M., et al., Genome-Wide CpG Island Profiling of Intraductal Papillary Mucinous Neoplasms of the Pancreas. Clinical Cancer Research, 2012. 18(3): p. 700-712.
[32′] Sato, N., et al., Aberrant methylation of CpG islands in intraductal papillary mucinous neoplasms of the pancreas. Gastroenterology, 2002. 123(1): p. 365-72.
[33′] Ideno, N., et al., Intraductal Papillary Mucinous Neoplasms of the Pancreas With Distinct Pancreatic Ductal Adenocarcinomas Are Frequently of Gastric Subtype. Annals of Surgery, 2013. 258(1): p. 141-151.
[34′] Hong, S.-M., et al., Multiple genes are hypermethylated in intraductal papillary mucinous neoplasms of the pancreas. Modern Pathology, 2008. 21(12): p. 1499-1507.
[35′] Hata, T., et al., Predicting the Grade of Dysplasia of Pancreatic Cystic Neoplasms Using Cyst Fluid DNA Methylation Markers. Clin Cancer Res, 2017. 23(14): p. 3935-3944.
[36′] Sato, N., et al., Frequent hypomethylation of multiple genes overexpressed in pancreatic ductal adenocarcinoma. Cancer Res, 2003. 63(14): p. 4158-66.
[37′] Singh, N., et al., Clinical significance of promoter methylation status of tumor suppressor genes in circulating DNA of pancreatic cancer patients. J Cancer Res Clin Oncol, 2020. 146(4): p. 897-907.
[38′] Henriksen, S. D., et al., Promoter hypermethylation in plasma-derived cell-free DNA as a prognostic marker for pancreatic adenocarcinoma staging. Int J Cancer, 2017. 141(12): p. 2489-2497.
[39′] Henriksen, S. D., et al., Cell-free DNA promoter hypermethylation in plasma as a predictive marker for survival of patients with pancreatic adenocarcinoma. Oncotarget, 2017. 8(55): p. 93942-93956.
[40′] Dauksa, A., et al., Whole Blood DNA Aberrant Methylation in Pancreatic Adenocarcinoma Shows Association with the Course of the Disease: A Pilot Study. PLoS ONE, 2012. 7(5): p. e37509.
[41′] Pietrasz, D., et al., Prognostic value of circulating tumour DNA in metastatic pancreatic cancer patients: post-hoc analyses of two clinical trials. British Journal of Cancer, 2022. 126(3): p. 440-448.
[42′] Yu, F., et al., CFEA: a cell-free epigenome atlas in human diseases. Nucleic Acids Res, 2020. 48(D1): p. D40-d44.
[43′] Luchini, C., et al., Liquid Biopsy as Surrogate for Tissue for Molecular Profiling in Pancreatic Cancer: A MetaAnalysis Towards Precision Medicine. Cancers, 2019. 11(8): p. 1152.
[44′] Manne, A., et al., Predictive Value of MUC5AC Signature in Pancreatic Ductal Adenocarcinoma: A Hypothesis Based on Preclinical Evidence. International Journal of Molecular Sciences, 2023. 24(9): p. 8087.
[45′] Manne, A., et al., Expression profiles of MUC5AC glycoforms in neoplastic and non-neoplastic pancreatic tissue. Journal of Clinical Oncology, 2023. 41(16_suppl): p. e16008-e16008.
[46′] Benson, K. K., et al., Understanding the Clinical Significance of MUC5AC in Biliary Tract Cancers. Cancers, 2023. 15(2): p. 433.
[47′] Manne, A., et al., Understanding the Clinical Impact of MUC5AC Expression on Pancreatic Ductal Adenocarcinoma. Cancers, 2021. 13(12): p. 3059.
[48′] Kaur, S., et al., A Combination of MUC5AC and CA19-9 Improves the Diagnosis of Pancreatic Cancer: A Multicenter Study. Am J Gastroenterol, 2017. 112(1): p. 172-183.
[49′] Yue, T., et al., Enhanced discrimination of malignant from benign pancreatic disease by measuring the CA 19-9 antigen on specific protein carriers. PLoS One, 2011. 6(12): p. e29180.
[50′] Yang, K. S., et al., Extracellular Vesicle Analysis Allows for Identification of Invasive IPMN. Gastroenterology, 2021. 160(4): p. 1345-1358.e11.
[51′] Gold, D. V., et al., Detection of Early-Stage Pancreatic Adenocarcinoma. Cancer Epidemiology Biomarkers & Prevention, 2010. 19(11): p. 2786-2794.
[52′] Gold, D. V., et al., PAM4 enzyme immunoassay alone and in combination with CA 19-9 for the detection of pancreatic adenocarcinoma. Cancer, 2013. 119(3): p. 522-528.
[53′] Luka, J., P. M. Arlen, and A. Bristol, Development of a serum biomarker assay that differentiates tumor-associated MUC5AC (NPC-1C ANTIGEN) from normal MUC5AC. J Biomed Biotechnol, 2011. 2011: p. 934757.
[54′] Nagata, K., et al., Mucin expression profile in pancreatic cancer and the precursor lesions. J Hepatobiliary Pancreat Surg, 2007. 14(3): p. 243-54.
[55′] Yonezawa, S., et al., Gene expression of gastric type mucin (MUC5AC) in pancreatic tumors: its relationship with the biological behavior of the tumor. Pathol Int, 1999. 49(1): p. 45-54.
[56′] Kanno, A., et al., The Expression of MUC4 and MUC5AC Is Related to the Biologic Malignancy of Intraductal Papillary Mucinous Neoplasms of the Pancreas. Pancreas, 2006. 33(4): p. 391-396.
[57′] Matsuyama, M., et al., Evaluation of pancreatic intraepithelial neoplasia and mucin expression in normal pancreata. J Hepatobiliary Pancreat Sci, 2012. 19(3): p. 242-8.
[58′] Lee, M. S. and S. Pant, Personalizing Medicine With Germline and Somatic Sequencing in Advanced Pancreatic Cancer: Current Treatments and Novel Opportunities. American Society of Clinical Oncology Educational Book, 2021(41): p. e153-e165.
[59′] Sohal, D. P. S., et al., Efficacy of Perioperative Chemotherapy for Resectable Pancreatic Adenocarcinoma. JAMA Oncology, 2021. 7(3): p. 421.
[60′] Sohal, D., et al., SWOG S1505: Results of perioperative chemotherapy (peri-op CTx) with mfolfirinox versus gemcitabine/nab-paclitaxel (Gem/nabP) for resectable pancreatic ductal adenocarcinoma (PDA). Journal of Clinical Oncology, 2020. 38(15_suppl): p. 4504-4504.
[61′] Ahmad, S. A., et al., Surgical Outcome Results From SWOG S1505: A Randomized Clinical Trial of mFOLFIRINOX Versus Gemcitabine/Nab-paclitaxel for Perioperative Treatment of Resectable Pancreatic Ductal Adenocarcinoma. Ann Surg, 2020. 272(3): p. 481-486.
[62′] Ghaneh, P., et al., Immediate surgery compared with short-course neoadjuvant gemcitabine plus capecitabine, FOLFIRINOX, or chemoradiotherapy in patients with borderline resectable pancreatic cancer (ESPAC5): a fourarm, multicentre, randomised, phase 2 trial. The Lancet Gastroenterology & Hepatology, 2022.
[63′] Labori, K. J., et al., Short-course neoadjuvant FOLFIRINOX versus upfront surgery for resectable pancreatic head cancer: A multicenter randomized phase-II trial (NORPACT-1). Journal of Clinical Oncology, 2023. 41(17_suppl): p. LBA4005-LBA4005.

What is claimed:

1. A system for predicting a treatment-related outcome for a patient after a cancer therapy (e.g., chemotherapy), including an overall survival outcome, the system comprising:
a processor; and
a memory having instructions stored thereon, wherein execution of the instructions causes the processor to:
receive, via the processor, a methylation signature comprising methylated nucleic acid sequences (e.g., DNA, cell-free DNA (cfDNA), or RNA) or RNA sequencing signature acquired from a sample of a patient for at least one gene selected from the group consisting of BNIP3, CES2, CHFR, CXCL5, GSTM2, ITGB4, MUC4, MUC5AC, ONECUT2, PRMT1, RUNX1, SFN, SLC22A3, SOX8, TACC3, KCNH2, PROKR2, IGF1R, and TMEM139;
determine, via a trained AI model, using the received sequences, an indicator corresponding to an overall survival outcome of the patient from pancreatic cancer and/or associated treatments; and
output the determined indicator via a report or graphical user interface, wherein the output is subsequently employed to direct or adjust treatment of the pancreatic cancer for the patient.

2. The system of claim 1, wherein the trained AI model was trained using sequences for a plurality of genes, including at least 5 of BNIP3, CES2, CHFR, CXCL5, GSTM2, ITGB4, MUC4, MUC5AC, ONECUT2, PRMT1, RUNX1, SFN, SLC22A3, SOX8, TACC3, KCNH2, PROKR2, IGF1R, TMEM139, wherein the methylation signature or RNA sequencing signature is stratified for a patient population having a high risk group label and a lower risk group label for overall survival.

3. The system of claim 1, wherein the overall survival is determined at 6 months, 1 year, or 2 years from date of diagnosis of the pancreatic cancer.

4. The system of claim 1, wherein execution of the instructions further causes the processor to additionally predict at least one of a predicted duration of response, a predicted progression-free survival time, and predicted time to progression.

5. The system of claim 4, wherein the instructions to determine the additional prediction for the at least one of the predicted duration of response, the predicted progression-free survival time, and the predicted time to progression includes:
instructions to determine, via a second trained AI model, the received methylated sequences or RNA sequences for at least one gene selected from the group consisting of BNIP3, CES2, CHFR, CXCL5, GSTM2, ITGB4, MUC4, ONECUT2, PRMT1, RUNX1, SFN, SLC22A3, SOX8, TACC3, IGF1R, KCNH2, MUC5AC, SLC22A2, SST, TMEM139, ISG15, PROKR2, SLC38A5, and SMARCA2.

6. The system of claim 4, wherein the instructions to determine the additional prediction for the predicted duration of response, includes:
instructions to determine, via a second trained AI model, the received methylated sequences or RNA sequences for at least one gene in a gene selected from the group consisting of
BNIP3, CES2, CHFR, CXCL5, GSTM2, ITGB4, MUC4, ONECUT2, PRMT1, RUNX1, SFN, SLC22A3, SOX8, TACC3, IGF1R, KCNH2, MUC5AC, SST, and TMEM139.

7. The system of claim 4, wherein the instructions to determine the additional prediction for the predicted progression-free survival time includes:
instructions to determine, via a second trained AI model, the received methylated sequences or RNA sequences for at least one gene in a gene selected from the group consisting of BNIP3, CES2, IGF1R, ISG15, ITGB4, KCNH2, ONECUT2, PROKR2, RUNX1, SFN, SLC22A3, SLC38A5, SMARCA2, SOX8, SST, and TACC3.

8. The system of claim 4, wherein the instructions to determine the additional prediction for the predicted time to progression includes:
instructions to determine, via a second trained AI model, the received methylated sequences or RNA sequences for at least one gene in a gene selected from the group consisting of BNIP3, CES2, CHFR, CXCL5, GSTM2, IGF1R, ISG15, ITGB4, KCNH2, MUC4, ONECUT2, PROKR2, RUNX1, SFN, SLC22A3, SLC38A5, SMARCA2, SOX8, SST, and TACC3.

9. The system of claim 5, wherein the predicted duration of response, the predicted progression-free survival time, and the predicted time to progression is determined at 6 months, 1 year, or 2 years from a date of diagnosis or a date of initial treatment.

10. The system of claim 1, wherein the trained AI model is a convolutional neural network.

11. The system of claim 1, wherein the methylated sequences or RNA sequences were acquired via a sequencing operation.

12. The system of claim 11, wherein the sequencing operation comprises an Enzymatic Methylation Sequencing operation.

13. The system of claim 1, wherein the sample comprises blood plasma and/or tissues.

14. The system of claim 1, wherein pancreatic cancer comprises pancreatic ductal adenocarcinoma (PDA).

15. The system of claim 4, wherein the instructions to determine the additional prediction for the at least one of the predicted duration of response, the predicted progression-free survival time, and the predicted time to progression includes:
instructions to determine, via a second trained AI model, the received methylated sequences or RNA sequences for at least one gene selected from the group consisting of ABCB1, ABCB4, ABCC1, ABCC10, ABCC3, ABCC5, ABCC6, ABCC8, ABCC9, ABCG2, ANGPTL4, ARID1A, ASXL2, ATM, BCL2L1, BICC1, BNIP3, BRCA1, CADM1, CD44, CES2, CHFR, CTNNB1, CTPS2, CXCL5, DCK, DKK3, DPYD, EGFR, EIF5A, ENO1, GLO1, GSDME, GSTM1, GSTM2, HMGA1, HNF1A, HSPA5, HSPB1, IGF1R, IGFBP3, ISG15, ITGA3, ITGB4, JAG1, KCNH2, LDHA, MAP2, MAP3K7, MCL1, METTL3, MLH1, MUC4, MUC5AC, NOTCH2, NRP1, NT5C1A, ONECUT2, PRMT1, PROKR2, PTGES2, PYCARD, RELL2, RRM1, RRM2, RRP9, RUNX1, SFN, SLC22A2, SLC22A3, SLC29A1, SLC2A1, SLC38A5, SMARCA2, SNRPF, SOX8, SST, TACC3, TET1, TFAM, TGM2, TMEM139, TPX2, TRIM31, TYMS, UBE2T, USP8, VASH2, YEATS4, and ZEB1.

16. The system of claim 1, wherein the trained AI model was trained using cfDNA gene methylation signature comprising the methylated sequences from isolated cfDNA from plasma of a patient.

17. A method for predicting a treatment-related outcome for a patient after a cancer therapy (e.g., chemotherapy), including an overall survival outcome, the method comprising:
receiving, via a processor, a methylation signature comprising methylated nucleic acid sequences (e.g., DNA, cell-free DNA (cfDNA), or RNA) or RNA sequencing signature acquired from a sample of a patient for at least one gene selected from the group consisting of BNIP3, CES2, CHFR, CXCL5, GSTM2, ITGB4, MUC4, MUC5AC, ONECUT2, PRMT1, RUNX1, SFN, SLC22A3, SOX8, TACC3, KCNH2, PROKR2, IGF1R, and TMEM139;
determining, via a trained AI model, using the received methylated sequences or RNA sequences, an indicator corresponding to an overall survival outcome of the patient from pancreatic cancer and/or associated treatments; and
outputting the determined indicator via a report or graphical user interface, wherein the output is subsequently employed to direct or adjust treatment of the pancreatic cancer for the patient.

18. The method of claim 17, wherein the trained AI model was trained using methylated sequences or RNA sequences for a plurality of genes, including at least 5 of BNIP3, CES2, CHFR, CXCL5, GSTM2, ITGB4, MUC4, MUC5AC, ONECUT2, PRMT1, RUNX1, SFN, SLC22A3, SOX8, TACC3, KCNH2, PROKR2, IGF1R, TMEM139, wherein the methylation signature or RNA sequencing signature is stratified for a patient population having a high risk group label and a lower risk group label for overall survival.

19. The method of claim 17, wherein the overall survival is determined at 6 months, 1 year, or 2 years from date of diagnosis of the pancreatic cancer.

20. A non-transitory computer-readable medium having instructions stored thereon, wherein execution of the instructions causes a processor to:
receive, via the processor, a methylation signature comprising methylated nucleic acid sequences (e.g., DNA, cell-free DNA (cfDNA), or RNA) or RNA sequencing signature acquired from a sample of a patient for at least one gene selected from the group consisting of BNIP3, CES2, CHFR, CXCL5, GSTM2, ITGB4, MUC4, MUC5AC, ONECUT2, PRMT1, RUNX1, SFN, SLC22A3, SOX8, TACC3, KCNH2, PROKR2, IGF1R, and TMEM139;
determine, via a trained AI model, using the received methylated sequences or RNA sequences, an indicator corresponding to an overall survival outcome of the patient from pancreatic cancer and/or associated treatments; and
output the determined indicator via a report or graphical user interface, wherein the output is subsequently employed to direct or adjust treatment of the pancreatic cancer for the patient.
